(19) United States
(12) Patent Application Publication
(10) Pub. No.: US 2010/0055675 A1
(43) Pub. Date: Mar. 4, 2010

(54) METHOD FOR DETECTING MEASLES VIRUS, MEMBRANE ASSAY TEST DEVICE, AND MEMBRANE ASSAY TEST KIT

(75) Inventors: Hiroshi KUMAMOTO, Tatsuno-shi (JP); Kouji Sakaguchi, Kobe-shi (JP); Ayumi Asaeda, Akashi-shi (JP); Takehiro Hasegawa, Kobe-shi (JP); Naoya Okitsu, Kobe-shi (JP); Noriaki Nakajima, Kobe-shi (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi (JP)

(21) Appl. No.: 12/550,102

(22) Filed: Aug. 28, 2009

(30) Foreign Application Priority Data
Aug. 29, 2008 (JP) 2008-222626
Oct. 24, 2008 (JP) 2008-274807
Aug. 12, 2009 (JP) 2009-187024

(51) Int. Cl. C12Q 1/70; C12N 5/10; C07K 16/00

(52) U.S. Cl. 435/5; 435/346; 530/388.1

(57) ABSTRACT

Method for detecting a measles virus in an analyte, comprising forming a complex of a first monoclonal antibody being capable of binding to a first epitope of a measles virus nuclear protein and being immobilized on a solid phase, a second monoclonal antibody being capable of binding to a second epitope of a measles virus nuclear protein different from the first epitope and being labeled, and a measles virus nuclear protein contained in the analyte, on the solid phase; and detecting the measles virus based on the amount of the label of the complex formed on the solid phase, is disclosed. Membrane assay test device, and membrane assay test kit are also disclosed.

### FIELD OF THE INVENTION

The invention relates to a method for detecting a measles virus by using an anti-measles virus monoclonal antibody capable of binding to a measles virus antigen. The invention also relates to a lateral flow type membrane assay test device for detecting a measles virus as well as a membrane assay test kit using the same. The invention also relates to a flow-through type membrane assay test kit for detecting a measles virus.

### BACKGROUND

Measles virus is a single-strand RNA virus belonging to of the genus Morbillivirus in the family Paramyxoviridae and having an envelope with a diameter of 100 to 250 nm. When infected with measles viruses, the measles viruses first grow in respiratory epithelial cells and then grow mainly in systemic lymphoid tissues in lymph nodes, the spleen and thymus gland, to develop lymphopenia and immunosuppression. Measles viruses are highly infective so that when humans are infected once with measles viruses, a secondary infection may be caused, thus making the examination of infections with measles viruses very important.

Known methods of examining the presence or absence of measles infection include methods of detecting antibodies against measles viruses present in the body of a subject. For example, WO93/22683 describes a method wherein a recombinant measles virus nuclear protein antigen containing at least one epitope to be recognized by measles-specific immunoglobulin IgM is contacted with a human serum sample, thereby detecting the presence of measles-specific immunoglobulin IgM bound to the antigen.

WO93/22683 describes a method of detecting a measles virus contained in an analyte sample. Specifically, this document describes a measles virus test agent wherein an anti-measles virus monoclonal antibody is immobilized on the surface of a micro-solid carrier as a virus-aggregating test agent wherein a molecular having affinity for a protein on a measles virus surface layer is immobilized on the surface of a micro-solid carrier.

However, the method described in WO93/22683 is problematic in the following respect. That is, even when infected with measles viruses, a time of at least 3 days is required until the antibody titer of IgM is increased in the body of an infected patient, and thus the infection with measles viruses cannot be detected at an early stage by the method described above. Moreover, if the examination by the method described above is conducted before the antibody titer of IgM is increased after infection with measles viruses, pseudo-negativity may result.

According to the method described in U.S. Pat. No. 6,060,254, the presence or absence of measles viruses themselves can be judged, but this method requires use of a positive control and a negative control, and thus the operation required in the examination is troublesome and measles infection cannot be promptly judged.

As described above, measles viruses are highly infective so that there is demand for early detection of measles infection and for preventive measures against secondary infection, by prompt examination of measles infection. The methods described in WO93/22683 and U.S. Pat. No. 6,060,254 supra failed to provide a method of examining measles infection, which satisfies the requirements in promptness.

### SUMMARY OF THE INVENTION

A first aspect of the invention is method for detecting a measles virus in an analyte, comprising: forming a complex of a first monoclonal antibody being capable of binding to a first epitope of a measles virus nuclear protein and being immobilized on a solid phase, a second monoclonal antibody being capable of binding to a second epitope of a measles virus nuclear protein different from the first epitope and being labeled, and a measles virus nuclear protein contained in the analyte, on the solid phase; and detecting the measles virus based on the amount of the label of the complex formed on the solid phase.

A second aspect of the invention is a test device for a membrane assay comprising: a membrane carrier containing a judgment zone that holds a first monoclonal antibody capable of binding to first epitope of a measles virus nuclear protein; and a label holding member that holds a second monoclonal antibody being capable of binding to second epitope of a measles virus nuclear protein different from the first epitope and being labeled.

A third aspect of the invention is a test kit for a membrane assay comprising: the test device for a membrane assay; and an analyte treatment liquid containing a nonionic surfactant and being mixed with an analyte to prepare a measurement sample.

A fourth aspect of the invention is a test kit for a membrane assay comprising: a test device provided with a membrane carrier containing a judgment zone holding a first monoclonal antibody capable of binding to first epitope of a measles virus nuclear protein; and a label solution containing a second monoclonal antibody being capable of binding to second epitope of a measles virus nuclear protein different from the first epitope and being labeled.

### BRIEF DESCRIPTION OF THE DRAWINGS

FIG. 1 is a skeleton framework of a test kit for lateral flow type membrane assay (for immunochromatography);

FIG. 2A is a plan view of a test device for the test kit for membrane assay in FIG. 1, and FIG. 2B is a side view thereof;

FIG. 3 is an exploded plain view of an analyte treatment container;

FIG. 4 is a diagram showing a usage state of the membrane assay test kit in FIG. 1;

FIG. 5 is a diagram showing another example of the membrane assay test device;

FIG. 6 is a skeleton framework of a test kit for flow-through type membrane assay; and

FIG. 7A is a plan view of the test device in FIG. 6, and FIG. 7B is a sectional view thereof along the arrow X-X.

FIG. 8 shows a diagram of epitope analysis.

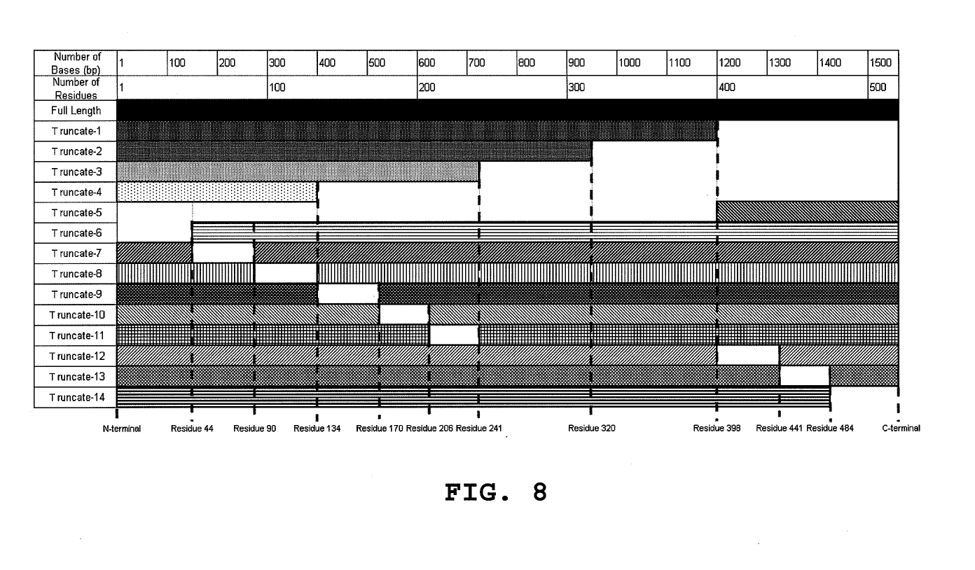

### DETAILED DESCRIPTION OF THE PREFERRED EMBODIMENTS

Hereinafter, embodiments of the test device of the invention will be described in detail with reference to the accompanying drawings.

In this specification, the “monoclonal antibody” includes fragments of the monoclonal antibody and derivatives thereof. Specific examples of the fragments of the monoclonal antibody and derivatives thereof include Fab fragments, F(ab′) fragments, F(ab)₂ fragments and sFv fragments. The subclass of the antibody is not limited to IgG, and may be IgM.

As the anti-measles virus monoclonal antibody, an antibody capable of binding to a nuclear protein of measles virus is used. The nuclear protein has higher antigenicity than that of other structural proteins such as an envelope and can be estimated to be contained in a larger amount in patients' analytes and is thus usable for improving the sensitivity in detection with the anti-measles virus monoclonal antibody.

The anti-measles monoclonal antibody can be obtained by immunizing an animal with a measles virus antigen by a known immunological method and then using the cells of the immunized animal to produce a hybridoma. Hybridoma MV2-2649 capable of producing antibody 2649 has been deposited under Accession No. NITE BP-563, hybridoma MV2-3241 capable of producing antibody 3241 under Accession No. NITE BP-564, hybridoma MV2-3707 capable of producing antibody 3707 under Accession No. NITE BP-565, and hybridoma MV3-320 capable of producing antibody 320 under Accession No. NITE BP-566, with NITE Patent Microorganisms Depositary, National Institute of Technology and Evaluation (NITE), National Institute of Advanced Industrial Science and Technology, Japan (accession date: Jul. 22, 2009).

The measles virus antigen can be obtained by purification from biological samples of measles-infected patients. The measles virus antigen can also be obtained by integrating a DNA encoding a measles virus nuclear protein in a plasmid, then introducing it into a host cell, and expressing it.

When an animal is immunized with the antigen, an adjuvant is preferably administered. By using the adjuvant, the immune response of the immunized animal to the antigen can be enhanced. The type of the adjuvant is not particularly limited, and for example, Freund complete adjuvant (FCA), Freund incomplete adjuvant (FIA), monophosphoryl lipid A (MPL, trade name: Ribi), trehalose dimycolate (TDM, trade name: RIBI), monophosphoryl lipid A and trehalose dimycolate mixture (MPL+TDM, trade name: RIBI Adjuvant System), Bordetella pertussis vaccine, muramyl dipeptide (MDP), aluminum adjuvant (ALUM) and the like are used. Among them, a plurality of adjuvants maybe used in combination. Preferably, FCA is used at the time of first immunization, and FIA or Ribi is used at the time of second and the subsequent immunization.

The schedule for immunization can be suitably altered depending on whether the adjuvant is administered or not and according to the administration route and the type of an animal to be immunized. Hereinafter, the immunization wherein a mouse is used as the immunized animal is described.

In the first immunization, an adjuvant-mixed measles virus antigen solution is injected intraperitoneally, subcutaneously or intramuscularly. The volume of the adjuvant-mixed measles virus antigen solution to be injected is preferably 0.05 to 1 mL, and the mass of the measles virus antigen contained therein is preferably 10 to 200 μg. When the adjuvant is not used, an increased amount of measles virus antigen may be intraperitoneally injected for immunization. Booster immunization is carried out once to 6 times every about 2 to 4 weeks after the initial immunization. About 1 to 4 weeks after the booster immunization, final immunization is carried out by intravenously injecting the measles virus antigen solution. After 3 to 5 days after the final immunization, spleen cells can be separated from the mouse to give antibody-producing cells.

The antibody-producing cells thus prepared are fused with myeloma cells. The origin of myeloma cells is not particularly limited, and those derived from a mouse, rat or human are used, but those derived from an animal of the same spices as the immunized animal are preferably used, and those derived from an animal of the same species and strain as the immunized animal are more preferably used. When a mouse is used as the origin of myeloma cells, it is preferable to use an established myeloma cell strain such as mouse myeloma P3X63-Ag8, P3X63-Ag8-U1, P3NS1-Ag4, SP2/o-Ag14 or P3X63-Ag8/653. Some myeloma cells produce an immunoglobulin light chain, and when such myeloma cell is used as a subject of fusion, this light chain may be bound to an immunoglobulin heavy chain produced by the antibody-producing cell. Accordingly, a myeloma cell not producing an immunoglobulin light chain, for example, P3X63-Ag8·653 or SP2/o-Ag14 is preferably used.

The method of producing a hybridoma by fusing the antibody-producing cell with the myeloma cell is not particularly limited, and a known method can be used. Examples include a method of using polyethylene glycol (PEG method), a method of using Sendai virus and a method of using an electric fusion apparatus. In the PEG method, spleen cells and myeloma cells may be suspended in a mixing ratio of from 1:1 to 10:1, preferably from 5:1 to 10:1 in a suitable medium or buffer containing about 30 to 60% PEG (average molecular weight 1000 to 6000) and then incubated for about 30 seconds to 3 minutes under the conditions of a temperature of about 25 to 37° C. and pH 6 to 8. After the incubation is finished, the cells can be washed to remove the PEG solution, suspended again in a hypoxanthine-thymidine medium (HT medium) or the like, then seeded in, for example, a microtiter plate and continued to be cultured.

The cells after fusion are cultured in a selective medium and selected for hybridoma. The selective medium is not particularly limited insofar as in the medium, the parent cell strain perishes and only the fusion cells can grow. Usually, a hypoxanthine-aminopterin-thymidine medium (HAT medium) is used. Selection of the hybridoma is initiated by exchanging a part of the medium, preferably about half of the medium, with the selective medium, usually 1 day after the fusion procedure, and culturing the cells for 7 to 10 days.

Whether the growing hybridoma produces a desired antibody or not can be confirmed by collecting a culture supernatant and performing an antibody titer assay. The antibody titer assay is not particularly limited and a known method can be used. For example, the above supernatant diluted serially is added to, and reacted with, an immobilized antigen and further reacted with a secondary antibody (anti-globulin antibody, anti-IgG antibody, anti-IgM antibody and the like) labeled with a fluorescence substance, an enzyme, or a radioisotope (RI), whereby the antibody produced in the supernatant can be detected and the antibody titer can be measured. By screening the culture supernatant in each well of a plate, a hybridoma producing the desired antibody can be obtained.

Then, a single clone is separated. The separation method is not particularly limited, and a known method can be used. Examples include a limiting dilution method, a soft agar method, a method of using a fluorescence-activated cell sorter, and the like. In the limiting dilution method, for example, a hybridoma colony is diluted serially with a medium so as to be about 1 cell/well, inoculated in a culture plate, and then cultured. After culture for 10 days, it is confirmed whether the objective monoclonal antibody in a culture supernatant of 1 colony/well is produced or not, whereby the objective hybridoma clone can be isolated. When the resulting hybridoma clone is frozen in the presence of a cryoprotectant such as about 10 w/v % dimethyl sulfoxide (DMSO), glycerin or the like and stored at −196° C. to −70° C., the hybridoma clone can be stored semi-permanently. The cells can be used after rapidly thawed in a thermostatic bath at about 37° C. at use. The cells are used preferably after they are sufficiently washed such that the cytotoxicity of the cryoprotectant does not remain.

For examining the immunoglobulin subclass of the antibody produced by the hybridoma, the hybridoma is cultured under general conditions, and the antibody secreted into its culture supernatant may be examined by using a commercially available kit for determination of antibody class/subclass.

The method used in obtaining a monoclonal antibody from the hybridoma is suitably selected depending on a necessary amount and the properties of the hybridoma. Examples include a method of obtaining a monoclonal antibody from ascitic fluid of a mouse transplanted with the hybridoma, a method of obtaining a monoclonal antibody from a culture supernatant obtained by cell culture, and the like. Insofar as the hybridoma can grow in the abdomen of the mouse, the monoclonal antibody can be obtained at a high concentration of several mg/ml from the ascitic fluid. The hybridoma not capable of growing in vivo is obtained from a culture supernatant in cell culture. The method of obtaining the monoclonal antibody in cell culture is advantageous over a method conducted in vivo in that although the amount of the antibody produced is low, formation of the antibody is easy with less contamination with immunoglobulins and other contaminants contained in the mouse abdomen.

When the monoclonal antibody is obtained from ascitic fluid of a mouse transplanted with the hybridoma, the hybridoma is transplanted in the abdomen of a mouse to which a substance possessing immunosuppressant properties, such as pristane (2,6,10,14-tetramethylpentadecane), has previously been administered, and after about 1 week, the ascitic fluid accumulated therein is collected. In the case of a hybridoma obtained by fusing cells from animals of different animal species (for example, mouse and rat), it is preferable to use a nude mouse and a radiation-treated mouse.

When the antibody is obtained from the cell culture supernatant, it is possible to use, for example, culture methods used in maintaining cells, such as a stationary culture method, a high-density culture method and a spinner flask culture method. By using any of these methods, the hybridoma is cultured to give a culture supernatant containing the antibody.

Purification of the monoclonal antibody from the ascitic fluid or culture supernatant can be carried out by using a known immunoglobulin purification method. The immunoglobulin purification method is not particularly limited, and examples include a fractionation by salting-out using ammonium sulfate or sodium sulfate, a PEG fractionation, an ethanol fractionation, a DEAE ion-exchange chromatographic method and a gel filtration method.

When the monoclonal antibody is mouse IgG, the antibody can be purified by affinity chromatography with a protein A-bound carrier or an anti-mouse immunoglobulin-bound carrier.

The anti-measles virus monoclonal antibody thus prepared can be used in immunoassay for detecting a measles virus contained in an analyte or a sample obtained using an analyte. The immunoassay for detection of a measles virus is based preferably on a sandwich method as the detection principle wherein primary and secondary antibodies against a measles virus are used thereby forming a complex containing the primary antibody immobilized on a solid phase, the labeled secondary antibody, and a measles virus. Accordingly, the primary anti-measles virus monoclonal antibody and the secondary anti-measles virus monoclonal antibody are those recognizing, and binding to, different sites of a measles virus nuclear protein.

Moreover, this invention provides a monoclonal antibody capable of recognizing amino acid sequences of the residue 135 to the residue 241 of measles virus nuclear protein and binding to the amino acid sequences (135-124 antibody). This invention further provides a monoclonal antibody capable of recognizing amino acid sequences of the residue 485 to the residue 525 of measles virus nuclear protein and binding to the amino acid sequences (485-525 antibody). This invention further provides a monoclonal antibody capable of recognizing amino acid sequences of the residue 91 to the residue 134 of measles virus nuclear protein and binding to the amino acid sequences (91-134 antibody). Nuclear protein, to which the monoclonal antibodies bind, may be a protein of SEQ ID No: 9.

In detection of a measles virus by the sandwich method, one of three types of monoclonal antibodies mentioned above is able to be used as a labeled antibody, others are able to be used as antibodies immobilized on a solid phase. Combination of the antibody is not limited. In the light of detecting sensitivity, it is preferred to use the 135-241 antibody or 485-525 antibody as antibody immobilized on a solid phase and use the 91-134 antibody as labeled antibody. 135-241 antibody and 485-525 antibody may be immobilized on a solid phase together.

In detection of a measles virus by the sandwich method, the solid phase on which the antibody is to be immobilized may be one capable of immobilizing the antibody by a method known in the art, and examples of such solid phase include known materials such as a membrane, beads, particles, nanoparticles, a test tube, and a microtiter plate. As a labeling substance for labeling the antibody, an enzyme, a radioisotope, a fluorescent labeling substance, colored particles and colloid particles can be used.

Among various immunoassays with the sandwich method as the detection principle, a membrane assay using a membrane as the solid phase is preferable particularly from the viewpoint of ease and promptness in examination. The membrane assay includes a lateral flow type membrane assay and a flow-through type membrane assay, to both of which the anti-measles monoclonal antibody in this embodiment is applicable.

The lateral flow type membrane assay is a method in which a sample is dropped onto a membrane containing a judgment region having a capturing substance immobilized thereon, and the sample is developed horizontally over the membrane, thereby detecting a measurement object captured in the judgment region. On the other hand, the flow-through type membrane assay is a method in which a sample containing a measurement object is dropped onto a membrane on which a capturing substance (antibody) for capturing the measurement object (antigen) has been immobilized, and the sample is passed vertically through the membrane, thereby detecting the measurement object captured on the surface of the membrane. In either method, the measurement object is labeled with a predetermined labeling substance, and thus the presence or absence of the measurement object can be confirmed by examining whether the label appears or not on the membrane.

Hereinafter, the test kit for membrane assay in this embodiment will be described with reference to the drawings.

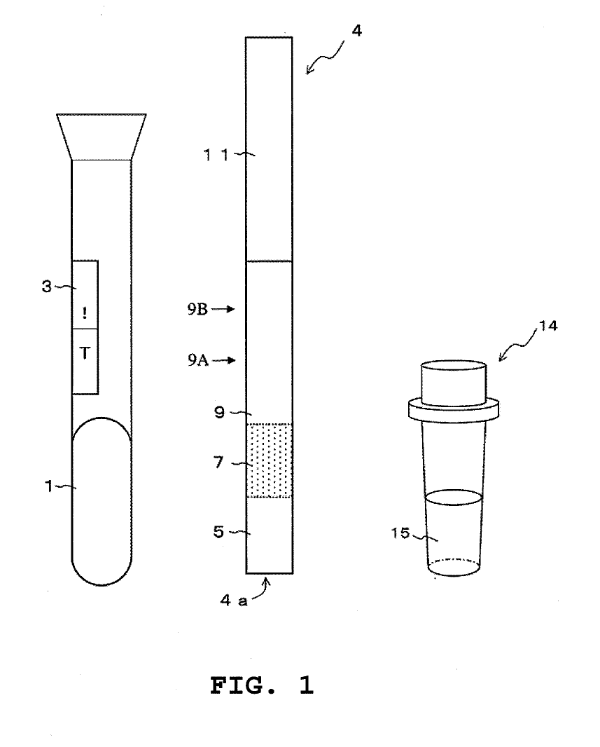

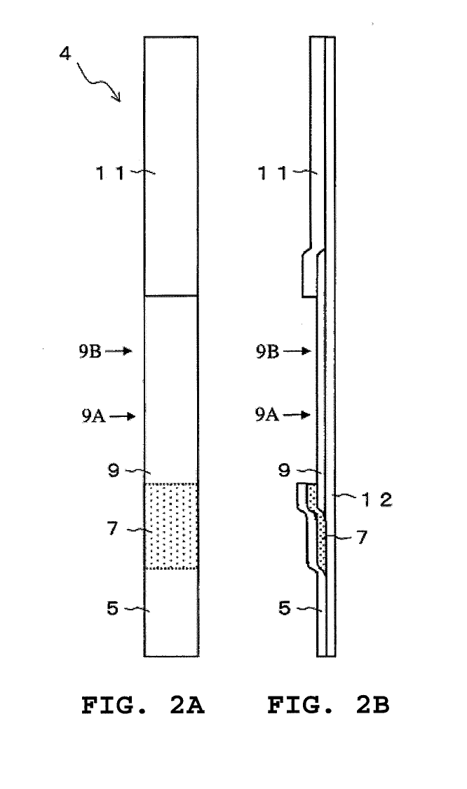

FIG. 1 is a diagram showing the outward appearance of the test kit for membrane assay (hereinafter referred to as “test kit”) in the first embodiment. This test kit is a test kit used in the lateral flow type membrane assay (lateral flow type immunochromatography) and includes a test container 1 for accommodating a sample, a membrane assay test device 4 used by inserting one side 4a into the test container 1 (hereinafter referred to as test device 4), and an analyte treatment container 14 in which an analyte treatment liquid 15 to be mixed with an analyte to prepare a measurement sample has been accommodated. FIG. 2A is a plan view of the test device 4 in FIG. 1, and FIG. 2B is a side view thereof.

As shown in FIG. 2, the test device 4 includes, on a substrate 12 consisting of a plastic plate having an adhesive layer thereon, a sample receiving member 5 consisting of a cotton nonwoven fabric, a label holding member 7 consisting of a glass fiber nonwoven fabric, a chromatographic membrane carrier 9 consisting of a nitrocellulose porous body (hereinafter referred to as chromatographic membrane carrier), and an absorbent member 11 consisting of a cellulose nonwoven fabric.

The label holding member 7 is arranged in contact with the sample receiving member 5 and holds an anti-measles virus monoclonal antibody being capable of binding specifically to a measles virus antigen and being labeled (hereinafter referred to “labeled antibody”), as well as a labeling substance for control. The labeled antibody is an anti-measles virus monoclonal antibody labeled with blue latex particles, while the labeling substance for control is streptavidin labeled with red latex particles. The chromatographic membrane carrier 9 is arranged in contact with the label holding member 7 and has a line-shaped judgment zone 9A and a control zone 9B in order from the upstream. An anti-measles virus monoclonal antibody capable of binding specifically to a measles virus antigen (hereinafter referred to as “capturing antibody”) has been immobilized on the judgment zone 9A, while biotin has been immobilized on the control zone 9B. The absorbent member 11 is arranged in contact with the chromatographic membrane carrier 9.

When a measles virus is contained in a sample, the labeled antibody held by the label holding member 7 recognizes a predetermined site of the measles virus and binds thereto via antigen-antibody reaction, thereby forming a complex. Then, the capturing antibody immobilized on the judgment zone 9A in the chromatographic membrane carrier 9 recognizes a different site of the measles virus, thereby capturing the complex. When the complex is captured, a blue line appears in the judgment zone 9A, whereby the measles virus can be visually detected.

Avidin is not captured by the capturing antibody in the chromatographic membrane carrier 9, but binds specifically to biotin, and thus the labeling substance for control is captured by biotin immobilized on the control zone 9B. When the labeling substance for control is captured, a red line appears in the control zone 9B, so it can be visually confirmed that the labeling substance for control has reached the control zone 9B. The control zone 9B is arranged downstream from the first judgment zone 9A, and thus it can be confirmed by the occurrence of a red line that the sample has passed through the first judgment zone 9A.

The test container 1 is composed of a cylindrical container in the form of a test tube with a bottom, which has a tapered receiving part 16 having an opening 1b and a sample accommodating part 17 for accommodating a sample in bottom 1a.

Label 3 is stuck on the outer wall of the test container 1. The label 3 has signs 24a and 24b in the test device 4 which indicate the judgment zone 9A and the control zone 9B in the test device 4, at positions corresponding to the judgment zone 9A and the control zone 9B in the test device 4 upon insertion into the test container 1. As shown in the diagram, the signs 24a and 24b in the label 3 are “T” and “!” respectively.

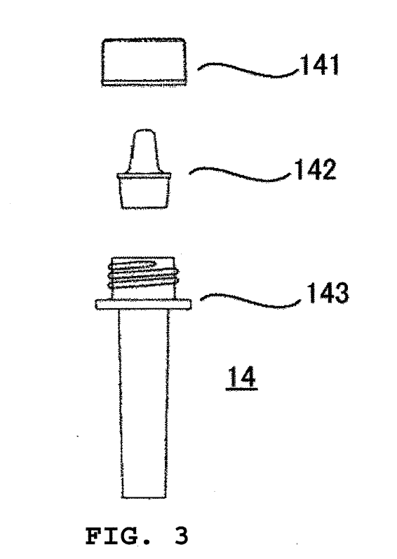

FIG. 3 is an exploded plain view of an analyte treatment container 14. As shown in the diagram, the analyte treatment container 14 is composed of a plastic bottle 141, nozzle 142 and cap 143. The bottle 141 accommodates an analyte treatment liquid 15 therein in such a state that the opening of the bottle 141 is closed with cap 143. The tip of the nozzle 142 is provided with a sample discharge opening, and a filter member is fitted to the inside of the nozzle.

The filter member fitted to the inside of the nozzle 142 comprises a first glass fiber filter, a second glass fiber filter with a larger membrane pore diameter than that of the first glass fiber filter and a nonwoven glass filter laminated in this order. This filter member is fitted to the nozzle 142 such that the glass filter is placed at the side of the nozzle 142 attached to the bottle 141 and the first glass fiber filter at the side of the sample discharge opening. The filter member is not limited to this constitution, but the nonwoven glass filter is preferably used to remove viscous components in an analyte, and one or two glass fiber filters are preferably used in this nonwoven glass filter.

The analyte treatment liquid 15 is preferably an aqueous solution containing a surfactant. Because the measles virus has an envelope, a surfactant is used to make openings in the envelope so that antigen proteins inside the virus are released into the analyte treatment liquid.

The type of the surfactant is not particularly limited, and a wide range of surfactants such as anionic surfactants, cationic surfactants, amphoteric surfactants and nonionic surfactants can be used.

The nonionic surfactants that can be used herein are preferably polyoxyethylene-based surfactants, more preferably ether-based surfactants. Specifically, it is preferable to use one or more members selected from the group consisting of polyoxyethylene alkyl phenyl ethers such as polyoxyethylene (9) octyl phenyl ether, polyoxyethylene (10) octyl phenyl ether and polyoxyethylene (9) nonyl phenyl ether, polyoxyethylene sorbitan fatty esters such as polyoxyethylene sorbitan monolaurate and polyoxyethylene sorbitan monooleate, a polyoxyethylene/polyoxypropylene copolymer, and a polyoxyethylene alkyl ether.

Although the amphoteric surfactant is not particularly limited, 3-[(3-cholamidopropyl)dimethylammonio]-1-propanesulfonate (CHAPS) or the like is preferably used. When the amount of the nonionic surfactant added to the analyte treatment liquid 15 is increased, the amphoteric surfactant may be used in combination therewith to improve the solubility and increase the storage stability of the analyte treatment liquid.

The anionic surfactant is not particularly limited, but it is preferable to use sodium dodecyl sulfate, sodium dodecyl sulfonate, sodium dodecyl-N-sarcosinate, sodium cholate, sodium dodecyl cholate, sodium taurodeoxycholate, and the like.

The analyte treatment liquid 15 preferably contains a thiocyanic acid-based compound in order to prevent unspecific reaction. The thiocyanic acid-based compound is not particularly limited insofar as it is a water-soluble thiocyanic acid-based compound such as a thiocyanic acid ester or a thiocyanate, in addition to thiocyanic acid (NHCS). The constituent of thiocyanate includes inorganic bases including metals such as sodium and potassium, and organic base ammonium salts. The thiocyanate also includes hydrates and solvates of these salts. Specific examples include sodium thiocyanate, potassium thiocyanate, ammonium thiocyanate, guanidine thiocyanate, and the like, among which potassium thiocyanate and guanidine thiocyanate are preferable.

The analyte treatment liquid 15 preferably contains a reducing agent to decrease the viscosity of highly viscous substances occurring in analytes (particularly, nasal discharges, aspirates from the nasal cavity, fluids wiped out of the nasal cavity and fluids wiped out of the pharynx). The reducing agent is preferably a sulfur-containing reducing compound and includes, for example, mercaptoethylamine, mercaptoethylamine hydrochloride, mercaptoethanol, dithiothreitol, cysteine, N-acetyl-L cysteine, S-2 aminoethylisothiourea dihydrobromide, tris(2-carboxyethyl)phosphine, hydrosulfite salt, sulfite salt and the like.

The analyte treatment liquid 15 may contain a chelating agent to suppress the activity of an enzyme decomposing an antigen protein or to reduce nonspecific reaction. The chelating agent can include, for example, ethylenediaminetetraacetic acid, 1,2-cyclohexanediaminetetraacetic acid, hexamethylenediaminetetraacetic acid, iminodiacetic acid, hydroxyethyliminodiacetic acid, 1,3-diaminopropan-2-oltetraacetic acid, diethylenetriaminepentaacetic acid, ethylenediaminediacetic acid, ethylenediaminediacetic acid dipropionic acid, ethylenebis(oxyethylenenitrilo)tetraacetic acid, ethylenediamine-tetrakis(methylenephosphonic acid), ethylenediaminedipropionic acid, hydroxyethylethylenediaminetriacetic acid, N-(2-hydroxylethyl)ethylenediaminetriacetic acid, nitrilotriacetic acid, nitrilotripropionic acid, nitrilotris(methylenephosponic acid), 2(hydroxyethyl)glycine and 1,2-diaminopropanetetraacetic acid, as well as salts thereof.

Alkali metal ions may be contained in the analyte treatment liquid 15. The alkali metal ions can be exemplified by lithium⁺ (Li⁺), sodium⁺ (Na⁺), potassium⁺ (K⁺), rubidium⁺ (Rb⁺), cesium⁺ (Cs⁺) and francium⁺ (Fr⁺), among which sodium and potassium can be preferably used. Alkali metal ions can be used alone or as a mixture of two or more thereof. Compounds capable of generating such alkali metal ions are not particularly limited, and for example, a mixture of one or more members selected from the group consisting of sodium chloride, potassium chloride, sodium hydroxide, potassium hydroxide, EDTA sodium salt, and sodium azide can be used. By adding alkali metal ions, nonspecific reaction can be suppressed. The content of alkali metal ions is 0.3 to 2.0 M, preferably 0.4 to 1.5 M, more preferably 0.45 M to 1.0 M.

The analyte treatment liquid 15 preferably contains a buffer, and examples of the buffer can include Good buffers such as MES, Bis-Tris, ADA, PIPES, ACES, MOPSO, BES, MOPS, TES, HEPES, DIPSO, TAPSO, POPSO, HEPPSO, EPPS, Tricine, Bicine, TAPS, CHES, CAPSO and CAPS, among which ADA, PIPES, ACES, MOPSO, BES, MOPS, TES, HEPES, DIPSO, TAPSO, POPSO, HEPPSO and EPPS are preferable, and PIPES, ACES, MOPSO, BES, MOPS, TES and HEPES are more preferable. The pH of the analyte treatment liquid is 5 to 10, preferably 5.5 to 9.0, more preferably 6.0 to 8.0.

The analyte mixed in the analyte treatment liquid 15 is not particularly limited as long as it is a biological sample collected from a subject. Examples of the analyte include, for example, blood, serum, urine, a nasal discharge, sputum, a nasal cavity swab, a nasal cavity aspirate and a throat swab. In a measles-infected patient, sputum, a nasal discharge, a nasal cavity swab or a throat swab in which measles viruses are easily detected is particularly preferable.

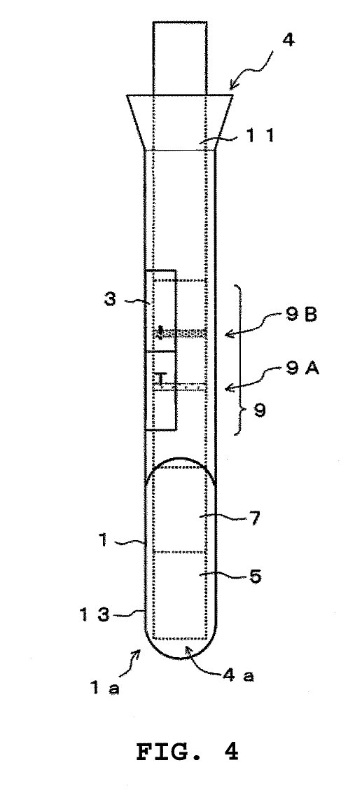

Hereinafter, a method of using the test kit in this embodiment will be described with reference to FIG. 4.

First, cap 143 of an analyte treatment container 14 is opened, and a collected analyte is added to bottle 141 and mixed with an analyte treatment liquid 15 to prepare a measurement sample 13. Thereafter, a nozzle 142 is fit, in place of the cap 143, to the opening of the bottle 141, and the measurement sample 13 is supplied via a filter member 44 from a sample discharge opening 46 to a test container 1. Then, one end 4a of a test device 4 is inserted into the test container 1 until the one end 4a is contacted with the bottom 1a of the test container 1 (It should be noted that herein termed “bottom” means a rounded portion of the test container 1). The test device 4 and the test container 1 are thereby aligned in the vertical direction. In this state, it is left as it is for about 10 to 20 minutes, the measurement sample 13 moves sequentially to the sample receiving member 5, the label holding member 7, the chromatographic membrane carrier 9 and the absorbent member 11 in this order by capillarity phenomenon. At the time when the measurement sample 13 passes through the label holding member 7, the labeling substances (the labeled anti-measles virus monoclonal antibody and the labeling substance for control) held by the label holding member 7 are eluted in the measurement sample. When a measles virus is contained in the measurement sample 13, a blue colored line appears in the judgment zone 9A by the action described above. Regardless of whether the virus occurs or not, a red line appears in the control zone 9B.

Hereinabove, the invention has been described with reference to a specific embodiment, but the test kit in this embodiment is not limited to such example and can be modified in various ways.

For example, the sample receiving member 5 can be made up of various materials such as glass fiber or cellulose fiber besides cotton. The label holding member 7 can be made up of cellulose fiber besides glass fiber. The labeled antibody and the labeling substance for control may be labeled with an enzyme, a fluorescent label, a magnetic label, a radioisotope, gold colloids or the like besides colored latex particles. The chromatographic membrane carrier 9 can be made up of various materials including not only nitrocellulose but also nylon (for example, nylon modified by introducing amino groups optionally substituted with a carboxyl group and an alkyl group), polyvinylidene difluoride (PVDF), and cellulose acetate. The absorbent member 11 can be made up of various materials such as glass fiber besides cellulose.

As described above, the sample receiving member 5, the label holding member 7, the chromatographic membrane carrier 9 and the absorbent member 11 can use those various structures besides a nonwoven fabric and a porous body which can develop a sample by capillary phenomenon.

In the embodiment illustrated above, the judgment zone 9A holds only one kind of measles virus monoclonal antibody, but the judgment zone is not limited to such constitution and may hold two or more anti-measles virus monoclonal antibodies. For example, the test device 4 may be produced by applying a sample containing two or more anti-measles virus monoclonal antibodies onto the chromatographic membrane carrier 9. By thus constituting the judgment zone holding two or more anti-measles monoclonal antibodies, the detection sensitivity of measles virus is improved, and consequently the possibility of false-negativity can be reduced. Two anti-measles virus monoclonal antibodies may be held separately on the chromatographic membrane carrier 9, thereby providing it with two or more judgment zones.

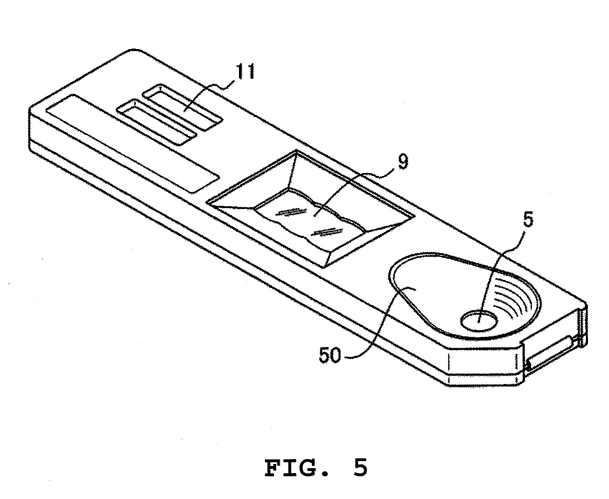

The embodiment illustrated above, the test container 1 for accommodating the measurement sample 13 is arranged, and the test device 4 is inserted into the test container 1 thereby performing examination, but the invention is not limited to such constitution, and the test container 1 may not be arranged. For example, the measurement sample 13 prepared using the analyte treatment liquid 15 may be dropped directly onto the sample receiving member 5. For this constitution, the test device 4 is preferably accommodated in a case having openings at positions corresponding to the sample receiving member 5, the chromatographic carrier 9 and the absorbent member 11 respectively. One example of the test device 4 thus constituted is shown in FIG. 5. By accommodating the test device 4 in the case 40 as shown in FIG. 5, the measurement sample can be prevented from leaking from each member of the test device 4 and can thus be hygienically examined. In addition, the liquid component contained in the measurement sample 13 easily evaporates through a plurality of openings arranged in the case 40, thereby accelerating the development of the measurement sample.

In the constitution shown in FIG. 5, the opening 50 arranged in the case 40 at a position corresponding to the sample receiving member 5 is formed in such a tapered shape that the area of the opening is decreased inwardly. By this constitution, the measurement sample dropped onto the sample receiving member 5 is retained in a predetermined amount on the opening 50 so that even if the measurement sample is dropped excessively, the measurement sample hardly overflows from the test device 40. Because the measurement sample is retained in a predetermined amount, the retained measurement sample is developed little by little on the test device 4, and thus the operation of dropping a small amount of the measurement sample little by little is not necessary, thus facilitating the examination.

Hereinabove, the test kit for lateral flow type membrane assay has been described, but the anti-measles virus monoclonal antibody in this embodiment can be applied to a test kit for flow-through type membrane assay. Hereinafter, the test kit for flow-through type membrane assay will be described with reference to the drawings.

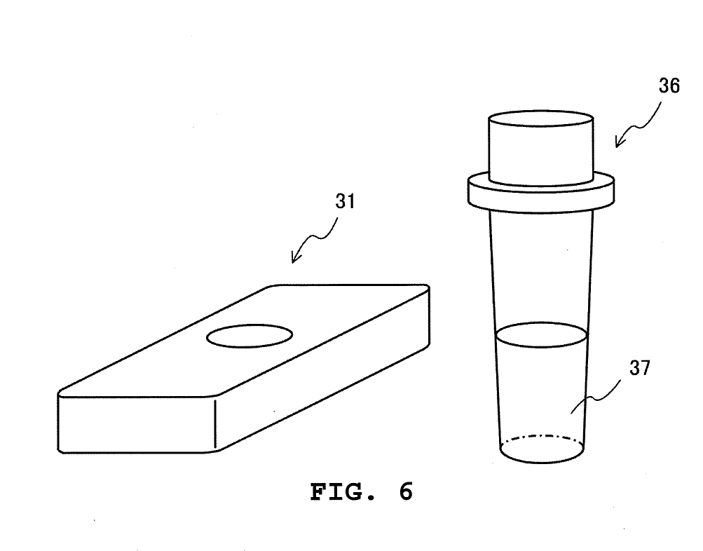

FIG. 6 is a view showing the test kit for membrane assay in the second embodiment. This test kit is a test kit used in flow-through type membrane assay and includes a test device 31 and an analyte treatment container 36 in which an analyte treatment liquid 37 to be mixed with an analyte to prepare a measurement sample has been accommodated.

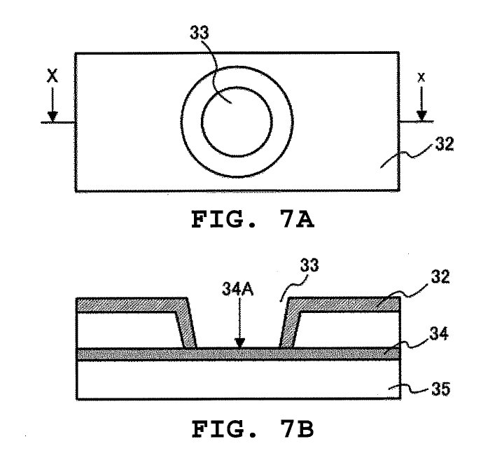

FIG. 7A is a plan view of the test device 31, and FIG. 7B is a sectional view thereof along the arrow X-X. The test device 31 comprises an absorbent member 35, a membrane carrier 34 and a cover member 32 laminated in this order from the lower layer. The cover member 32 has an opening 32 via which the judgment zone 34A of the membrane carrier 34 arranged in the lower layer is exposed. The judgment zone 34A supports a capturing antibody.

An analyte treatment container 36 has the same constitution as that of the analyte treatment container 14 in the first embodiment. An analyte treatment liquid 37 accommodated in the analyte treatment container 36 contains the same components as in the analyte treatment liquid 15 in the first embodiment, and the analyte treatment liquid 37 in this embodiment further contains a labeled antibody.

Hereinafter, a method of using the test kit in this embodiment will be described.

First, an analyte is suspended in the analyte treatment liquid 37 to prepare a measurement sample, as described in the first embodiment. When a measles virus is contained in the analyte, the measles virus binds, via antigen-antibody reaction, to the labeled antibody contained in the analyte treatment liquid 37, thereby forming a complex consisting of the measles virus/labeled antibody in the measurement sample.

Then, the measurement sample thus prepared is dropped in a predetermined amount onto the opening 33 of the test device 31, in the same manner as in the first embodiment described above. In this state, it is left as it is for about 20 minutes, the measurement sample is passed through the membrane carrier 34 and absorbed onto the absorbent member 35 arranged therebelow. When a measles virus is contained in the measurement sample, the capturing antibody immobilized on the judgment zone 34A in the membrane carrier 34 recognizes that site of the measles virus that is different from the binding site of the measles virus to the labeled antibody, and binds to the measles virus, thereby forming a complex consisting of the labeled antibody/measles virus/capturing antibody on the judgment zone 34A. A blue line thereby appears in the judgment zone 34A. Accordingly, the measles virus can be detected by visually judging whether a blue line appears on the judgment zone 34A.

In this embodiment illustrated above, the labeled antibody and the analyte treatment solution 37 are accommodated in the same container, but the invention is not limited to such constitution. For example, the labeled antibody and the analyte treatment liquid 37 may be accommodated in separate containers. Alternatively, the labeled antibody may be held by the membrane carrier 34, so as to be releasable by the liquid component contained in the measurement sample.

According to this embodiment, there can be provided a test kit capable of detecting a measles virus easily and rapidly with the anti-measles virus monoclonal antibody, as described above. Moreover, the measles virus can be detected and examined easily with a fewer number of parts, that is, by the test container 1, the test device 4 and the analytes treatment container 14 in the test kit for membrane assay in the first embodiment or by the test device 31 and the analyte treatment container 36 in the test kit for membrane assay in the second embodiment.

### Examples

### 1. Preparation of Monoclonal Antibody

### 1-1. Preparation of Measles Virus Nuclear Proteins

From RNA extracted from each wild measles virus strain, cDNA was synthesized by reverse transcription reaction. From each cDNA thus synthesized, a DNA product for measles virus nuclear protein was synthesized by polymerase chain reaction (PCR). Using ENDEXT (registered trade mark) wheat germ expression TRI-GG kit (Cell Free Science), the DNA product was integrated in a vector attached to the kit, to express measles virus nuclear proteins, thereby preparing an antigen solution. Using a sequencer (manufactured by Applied Biosystems) and sequence analysis software (manufactured by Hitachi Software), gene sequence analysis and predicted amino acid sequence analysis were performed. Amino acid sequences of the measles virus nuclear proteins thus obtained are set forth in SEQ ID NOS: 1 to 9 in the Sequence Listing. Hereinafter, the measles virus nuclear protein consisting of the amino acid sequence of SEQ ID NO: 1 will be referred to as Ag1 and the measles virus nuclear protein consisting of the amino acid sequence of SEQ ID NO: 2 will be referred to as Ag2; in this manner, the measles virus nuclear protein consisting of the amino acid sequence of SEQ ID NO: N will be referred to as AgN.

### 1-2. Immunization of Mice

100 μL of Freund complete adjuvant (FCA) was added to 100 μL phosphate-buffered saline (PBS) containing 60 μg of a measles virus strain (trade name: Rubeola (measles) Virus Infected Cell Extract) purchased from Advanced Biotechnologies Inc. (hereinafter ABI) and then emulsified to prepare 200 μL of FCA-mixed measles virus antigen solution. Separately, 200 μL of FIA-mixed measles virus antigen solution was prepared in the same manner as above except that Freund incomplete adjuvant (FIA) was used in place of FCA.

First immunization was carried out by intraperitoneally administering 200 μL of the FCA-mixed measles virus antigen solution to a 7-week-old female BALB/c mouse. After the first immunization, booster immunization with 200 μL of the FIA-mixed measles virus antigen solution was carried out every 2 weeks. The final immunization was carried out without using an adjuvant by intravenously injecting 500 μL of PBS containing 50 μg of Ag5 prepared in 1-1 in place of the measles virus strain manufactured by ABI (Immune Experiment 1).

The mouse was immunized in the same manner as in the immune experiment 1 except that as the immune antigen, Ag5 or Ag 8 prepared in 1-1 was used in place of the measles virus strain manufactured by ABI (Immune Experiment 2).

Four days after the final immunization, the spleen cells were separated and then fused with P3X63-Ag8 653 mouse myeloma cells by the PEG method to prepare a hybridoma.

### 1-2. Culture of the Hybridoma

The hybridoma was suspended at 2.5×10⁶ cells/ml in HAT medium and pipetted to each well of a 96-well plate (hereinafter, referred to as culture plate, manufactured by Corning) at 2.5×10⁵ cells/well. The culture plate was left in a thermostatic chamber at 37° C. in 8% CO₂ to initiate culture of the hybridoma. On the next day, 25 μg HAT medium was added to each well of the culture plate, and culture was further continued. When hybridoma colonies appeared by culture for 10 days, screening of a hybridoma producing the monoclonal antibody was carried out.

### 1-3. Screening of the Hybridoma

A measles virus (manufactured by ABI) diluted to a protein concentration of 0.5 μg/ml was added to 0.1 M phosphate-buffered saline (PBS, pH 7.5) containing 0.1 w/v % NaN₃, to prepare a measles virus antigen solution for immobilization. 100 μL of the measles virus antigen solution for immobilization was pipetted into each well of a 96-well plate (manufactured by NUNC, hereinafter, referred to as antigen-immobilized plate). The plate was left overnight at 4° C. and then washed 3 times with PBS containing Tween 20 at a concentration of 0.05% (hereinafter, referred to as first buffer solution). After washing, 300 μL of PBS containing BSA at a concentration of 1 w/v % (hereinafter, referred to as second buffer solution) was added to each well of the antigen-immobilized plate and left for 4 hours or more at 2 to 8° C. The antigen-immobilized plate was stored at 2 to 8° C. until use.

75 μL of the second buffer solution was added to each well of the antigen-immobilized plate. The hybridoma culture supernatant prepared in 1-3 above was removed from each well of the culture plate and then added at a volume of 25 μl/well to each well of the antigen-immobilized plate. After the second buffer solution and the culture supernatant were added, the antigen-immobilized plate was incubated at room temperature for 1 hour. After incubation, each well of the antigen-immobilized plate was washed with 300 μL of the first buffer solution. After washing, 100 μL of horseradish peroxidase-labeled anti-mouse Ig polyclonal antibody (Code No. P0447, manufactured by DAKO) diluted 10000-fold with the second buffer solution was added to each well of the antigen-immobilized plate. The plate was incubated at room temperature for 30 minutes, and then each well of the antigen-immobilized plate was washed with 300 μL of the first buffer solution. After washing, 100 μL of a substrate solution containing ortho-phenylene diamine (OPD) as a substrate of POD was added to each well and then left at room temperature for 10 minutes. Then, 100 μL of a reaction termination solution containing 2 N H₂SO₄ was added to each well of the antigen-immobilized plate. The reaction solution in each well was measured for its absorbance at 492 nm with a microplate reader (manufactured by Molecular Devices).

As a result, it has been confirmed that the prepared hybridoma produces an anti-measles virus monoclonal antibody.

### 2. Confirmation of the Reactivity of the Anti-Measles Virus Monoclonal Antibody

The reactivity of the anti-measles virus monoclonal antibody produced by the hybridoma prepared in 1 above was confirmed by the following experiment.

### 2-1. Reactivity Test

Antigen solutions containing Ag1 to Ag9 prepared in 1.1 were diluted 10-fold with the second buffer solution respectively.

A Sepharose beads/anti-mouse IgG antibody suspension in which Sepharose beads (manufactured by Amersham Biosciences) to which a commercial anti-mouse IgG antibody had been bound were contained at a density of 15 v/v % was prepared. This Sepharose beads/anti-mouse IgG antibody suspension, each of antigen dilutions prepared by diluting antigen solutions containing Ag1 to Ag9, to a concentration of 0.2 to 0.5 μg/mL with the second buffer solution, and the second buffer solution, were mixed in equal volumes to prepare antigen samples.

60 μL of the antigen sample and 30 μL antibody solution containing anti-MV mAb in this example diluted to an antibody concentration of 1.0 μg/mL with the second buffer solution were added to each well of a V-shaped 96-well plate (manufactured by Sanplatec Co., Ltd.). Then, the V-shaped 96-well plate was stirred at room temperature for 60 minutes and then left for 10 minutes to sediment the Sepharose beads.

Then, a microtiter plate to which an anti-measles virus monoclonal antibody was conjugated was prepared in the following manner. As the anti-measles virus monoclonal antibody conjugated to a microtiter plate, antibody 225 produced by hybridoma MV1-225 was used.

An antibody solution containing antibody 225 was diluted to a concentration of 10 μg/mL with 0.1 M phosphate-buffered saline (PBS, pH 7.5) containing 0.1 w/v % NaN₃. The resulting antibody 225 dilution was pipetted in a volume of 100 μL to each well of a microtiter plate (manufactured by NUNC) and left overnight at 4° C. Each well was washed 3 times with the first buffer solution, and then 300 μL of the second buffer solution was pipetted into each well, thereby blocking the antibody 255 in each well of the plate (hereinafter, this microtiter plate will be referred to as antibody 225-conjugated plate). After blocking, the plate was left at 2 to 8° C. for 4 hours or more in a stationary state. The antibody 225-conjugated plate was stored at 2 to 8° C. until use.

By Sysmex Corporation, hybridoma MV1-225 has been deposited with NITE Patent Microorganisms Depositary, National Institute of Technology and Evaluation (NITE), National Institute of Advanced Industrial Science and Technology, Japan, and assigned with Accession No. NITE AP-599.

Then, a biotin-labeled anti-measles virus monoclonal antibody solution and a streptavidin-labeled peroxidase (POD) solution were mixed to concentrations of 0.25 μg/mL and 40 mU/mL, respectively, with the second buffer solution, to prepare a POD-labeled antibody solution.

As the anti-measles virus monoclonal antibody to be labeled with biotin, antibody 1117 produced by hybridoma MV1-1117 was used. By Sysmex Corporation, hybridoma MV1-1117 has deposited with NITE Patent Microorganisms Depositary, National Institute of Technology and Evaluation (NITE), National Institute of Advanced Industrial Science and Technology and assigned with Accession No. NITE AP-600.

The second buffer solution in the antibody 225-conjugated plate was removed. After removal, 50 μL of the POD-labeled antibody solution and 50 μL of the supernatant of the reaction solution on the V-shaped 96-well plate were added to each well of the antibody 225-conjugated plate and then stirred at room temperature for 60 minutes. Thereafter, each well of the antibody 225-conjugated plate was washed 3 times with the second buffer solution.

Depending on the reactivity, with the antigen, of the anti-measles virus monoclonal antibody in this example, there are cases where the antigen is contained or not contained in the supernatant of the reaction solution on the V-shaped 96-well plate.

That is, when the anti-measles virus monoclonal antibody in this example is reactive with the antigen (that is, the antibody is bound to the antigen), a complex consisting of Sepharose bead/anti-mouse IgG-anti-measles virus monoclonal antibody/antigen is formed in the reaction solution. Due to the weight of the Sepharose bead, this complex is sedimented on each well of the V-shaped 96-well plate, and thus the supernatant does not contain the antigen. On the other hand, when the anti-measles virus monoclonal antibody in this example is not reactive with the antigen (that is, the antibody does not bind to the antigen), the above complex is not formed in the reaction solution, the antigen is not sedimented, and thus the supernatant contains the antigen.

Accordingly, when the anti-measles virus monoclonal antibody in this example does not show reactivity with the antigen, a complex consisting of the antibody 225/antigen/POD-labeled antibody is formed on well of the antibody 225-conjugated plate by the operation described above. When the anti-measles virus monoclonal antibody in this example shows reactivity with the antigen, the above complex is not formed.

Then, 100 μL of a substrate solution containing OPD being a substrate of POD was added to each well of the antibody 225-conjugated plate and left at room temperature for 10 minutes. Thereafter, 100 μL of a reaction termination solution (containing 2 N H₂SO₄) was added to each well of the antibody 225-conjugated plate, and the reaction solution in each well was measured for its absorbance at 492 nm with a microplate reader (manufactured by Molecular Devices). The absorbance thus obtained is referred to as absorbance A.

As is evident from the above description, absorbance A indicates the presence or absence of the complex on well of the antibody 225-conjugated plate. That is, lower absorbance is indicative of fewer conjugates, thus showing higher reactivity, with the antigen, of the anti-measles virus monoclonal antibody in this example.

In a control experiment, a control sample in which the 10 μg/mL antibody solution had been changed to the second buffer solution was prepared and examined in the same experiment as described above to determine its absorbance. The absorbance thus obtained is referred to as absorbance B.

In another control experiment, a control sample in which the 10 μg/mL antibody solution and the antigen sample had been changed to the second buffer solution was prepared and examined in the same experiment as described above to determine its absorbance. The absorbance thus obtained is referred to as absorbance C.

Using the absorbances A to C thus obtained, the absorptivity of each of the measurement samples obtained using the anti-measles virus monoclonal antibodies obtained in this example was determined according to formula (1) below. The obtained absorptivity is shown in Table 1, and the evaluation result of reactivity based on the absorptivity is shown in Table 2. In Table 2, “+++” indicates that the absorptivity determined by the formula (1) is 90% or more; “++”, 50% or more; “+”, 30% or more; and “−”, 30%.

Absorptivity (%)={(1−(A−C)/(B−C)}×100 (1)

TABLE 1

| | | Used antigens | | | | | | | | | |
|---|---|---|---|---|---|---|---|---|---|---|---|
| Monoclonal | | | | | | | | | | | Antigen |
| antibody | | | | | | | | | | | species |
| Name of | | Recombinant measles virus nuclear proteins | | | | | | | | | Type |
| antibody | | H1 | D3 | H1 | H1 | H1 | H1 | D9 | D5 | A | Name of |
| clone | Isotype | Ag1 | Ag2 | Ag3 | Ag4 | Ag5 | Ag6 | Ag7 | Ag8 | Ag9 | antigen |
| MV2-2649 | IgG1/κ | 88.0 | 92.2 | 87.1 | 100.0 | 97.8 | 96.9 | 85.6 | 89.2 | 82.0 | Absorptivity |
| MV2-3707 | IgG1/κ | 57.4 | 93.1 | 97.2 | 100.0 | 69.5 | 99.1 | 89.7 | 100.0 | 100.0 | |
| MV2-3241 | IgG2a/κ | 55.1 | 94.3 | 97.2 | 100.0 | 74.2 | 100.0 | 92.5 | 100.0 | 100.0 | |
| MV3-320 | IgG2b/κ | 100.0 | 99.6 | 100.0 | 100.0 | 100.0 | 100.0 | 100.0 | 100.0 | 100.0 | |

TABLE 2

| | | Used antigens | | | | | | | | | |
|---|---|---|---|---|---|---|---|---|---|---|---|
| Monoclonal | | | | | | | | | | | Antigen |
| antibody | | | | | | | | | | | species |
| Name of | | Recombinant measles virus nuclear proteins | | | | | | | | | Type |
| antibody | | H1 | D3 | H1 | H1 | H1 | H1 | D9 | D5 | A | Name of |
| clone | Isotype | Ag1 | Ag2 | Ag3 | Ag4 | Ag5 | Ag6 | Ag7 | Ag8 | Ag9 | antigen |
| MV2-2649 | IgG1/κ | ++ | +++ | +++ | +++ | +++ | +++ | ++ | +++ | ++ | Reactivity |
| MV2-3707 | IgG1/κ | ++ | +++ | +++ | +++ | ++ | +++ | ++ | +++ | +++ | |
| MV2-3241 | IgG2a/κ | ++ | +++ | +++ | +++ | ++ | +++ | +++ | +++ | +++ | |
| MV3-320 | IgG2b/κ | +++ | +++ | +++ | +++ | +++ | +++ | +++ | +++ | +++ | |

As shown in Tables 1 and 2, the antibody 2649, antibody 3707, antibody 3241 and antibody 320 obtained in this example showed high reactivity with all of the measles virus nuclear proteins Ag1 to Ag9.

From this result, the anti-measles virus monoclonal antibodies in this example can bind to the measles virus nuclear proteins and do not reduce the reactivity with the nuclear proteins regardless of the substitution mutation of a part of the amino acid sequence thereof.

### 3. Screening of the Binding Site of the Anti-Measles Virus Monoclonal Antibody to the Measles Virus Antigen

The binding site of the anti-measles virus monoclonal antibody in this example to the measles virus nuclear protein was confirmed by the following experiment.

### 3-1. Preparation of an Antigen Sample

An antigen solution containing the measles virus nuclear protein Ag3 prepared in 1-1 above was diluted to 0.15 μg/mL with the second buffer solution, to prepare an antigen sample containing Ag3.

### 3-2. Inhibition Test

An antibody 225-conjugated plate was prepared in the same manner as described in 2-1 above. 100 μL of the antigen sample containing Ag 3 was added to each well of the antibody 225-conjugated plate and stirred for 30 minutes. Thereafter, each well of the antibody 225-conjugated plate was washed 3 times with the first buffer solution.

A biotin-labeled antibody 3707 solution and a streptavidin-labeled peroxidase (POD) solution were mixed to 0.5 μg/mL and 40 mU/mL, respectively, with the second buffer solution, to prepare a POD-labeled antibody solution. 50 μL antibody solution prepared by diluting the antibody 2649 obtained in this example to a concentration of 10 μg/mL with the second buffer solution, and 50 μL of the POD-labeled antibody solution, were added to each well of the antibody 225-conjugated plate and then stirred at room temperature for 60 minutes. After stirring, the antibody 225-conjugated plate was washed 3 times with the first buffer solution.

Then, 100 μL of a substrate solution containing OPD was added to each well of the antibody 225-conjugated plate and then left at room temperature for 10 minutes. Then, 100 μL of a reaction termination solution (containing 2 N H₂SO₄) was added to each well of the antibody 225-conjugated plate, and the reaction solution in each well was measured for its absorbance at 492 nm with a microplate reader. The absorbance thus obtained is referred to as absorbance D.

In a control experiment, a control sample in which the 10 μg/mL antibody solution had been changed to the second buffer solution was prepared and examined in the same experiment as described above to determine the absorbance. The absorbance thus obtained is referred to as absorbance E.

In another control experiment, a control sample in which the 10 μg/mL antibody solution and the antigen sample had been changed to the second buffer solution was prepared and examined in the same experiment as described above to determine the absorbance. The absorbance thus obtained is referred to as absorbance F.

Using the absorbances D to F thus obtained, the absorptivity of each of the measurement samples in the experimental example was determined according to formula (2) below. On the basis of the obtained absorptivity, the homology between the binding sites of the antibodies 2649 and 3707 to the measles protein nuclear protein was evaluated.

The obtained absorptivity is shown in Table 3, and the evaluation result of homology based on absorptivity is shown in Table 4. In Table 4, “+++” indicates that the homology determined by the formula (2) is 90% or more; “++”, 50% or more; “+”, 30% or more; and “−”, 30%.

In the above experimental example, higher absorptivity as determined by (2) is indicative of higher homology between the binding sites of the antibodies 2649 and 3707 to the measles virus nuclear protein.

Absorptivity (%)={(1−(D−F)/(E−F)}×100 (2)

This experiment was conducted for every combination of an antibody contained in 10 μg/mL antibody solution in the experimental example and a biotin-labeled antibody, wherein the antibodies used in combination are selected from the antibody 2649, antibody 3707, antibody 3241 and antibody 320.

TABLE 3

| Antibody | | Labeled antibody | | | | |
|---|---|---|---|---|---|---|
| Name of antibody | | | | | | Name of |
| clone | Isotype | MV2-2649 | MV2-3707 | MV2-3241 | MV3-320 | clone |
| MV2-2649 | IgG1/κ | 83.8 | 16.6 | 17.7 | 81.6 | Absorptivity |
| MV2-3707 | IgG1/κ | −3.1 | 93.2 | 100.0 | −0.3 | |
| MV2-3241 | IgG2a/κ | 8.4 | 91.4 | 99.3 | −3.6 | |
| MV3-320 | IgG2b/κ | 54.7 | 56.4 | 48.7 | 99.6 | |

TABLE 4

| Antibody | | Labeled antibody | | | | |
|---|---|---|---|---|---|---|
| Name of antibody | | | | | | Name of |
| clone | Isotype | MV2-2649 | MV2-3707 | MV2-3241 | MV3-320 | clone |
| MV2-2649 | IgG1/κ | ++ | − | − | ++ | Reactivity |
| MV2-3707 | IgG1/κ | − | +++ | +++ | − | |
| MV2-3241 | IgG2a/κ | − | +++ | +++ | − | |
| MV3-320 | IgG2b/κ | ++ | ++ | + | +++ | |

From the results in Tables 3 and 4, the antibodies 2649 and 3707 are low in homology between their binding sites to the measles virus nuclear protein, and the both antibodies were suggested to be antibodies capable of recognizing, and binding to, different sites of the measles virus nuclear protein. This also applies to the combination of antibody 2649 and antibody 3241, the combination of antibody 320 and antibody 3707, and the combination of antibody 320 and antibody 3241.

From the results described above, it has been revealed that when the anti-measles virus monoclonal antibodies in this example are used in a specific combination, the combined anti-measles virus monoclonal antibodies bind to first and second (that is, different) binding sites of the measles virus nuclear protein respectively, thereby enabling the detection of the measles virus by the sandwich assay.

### 4. Preparation of a Test Device

The anti-measles virus monoclonal antibodies in this example were used to prepare the test device for lateral flow type membrane assay shown in FIG. 1.

Test Device 1 As the first monoclonal antibody held by the label holding member 7, the antibody 320 was used. As the second monoclonal antibodies held by the judgment zone 9A in the chromatographic membrane carrier 9, the antibodies 2649 and 3707 were used.

As shown in FIG. 1, an antibody solution containing the antibody 3707 diluted to a concentration of 2.0 mg IgG/mL with a phosphate buffer solution (pH 7.0) was applied to a width of 1 mm via an antibody coater (BioDot) onto the judgment zone 9A in the chromatographic membrane carrier 9 consisting of a nitrocellulose membrane, and then the antibody solution containing the antibody 2649 was applied in the same manner as above, onto a position apart by 1 mm from the previously applied position, and then dried at 50° C. for 30 minutes.

After drying, the chromatography membrane carrier 9 was immersed into a phosphate buffer solution (pH 7.0, containing BSA) to immobilize the antibodies on the chromatographic membrane carrier 9. Thereafter, the chromatography membrane carrier 9 was washed with a cleaning fluid (phosphate buffer solution, pH 7.0, containing SDS), and then dried at 40° C. for 120 minutes, to give a chromatographic membrane carrier 9.

Then, the antibody 320 was conjugated to blue colored polystyrene latex particles (particle diameter of 0.3 μm) and suspended in a buffer solution for dispersion (phosphate buffer solution, pH 7.0, containing BSA and sucrose), to prepare an antibody 320-conjugated latex particle suspension. The concentration of the antibody 320 was at 200 μg IgG per mL of 1% latex particle suspension. This antibody 320-conjugated latex particle suspension was added to a glass fiber pad and dried with a vacuum dryer to give a label holding member 7.

As the sample receiving member 5, the absorbent member 11 and the substrate 12, the members described in the above embodiment were used to obtain a test device 1.

Separately, a test device 2 was obtained in the same manner as described above except that as the antibody held by the judgment zone 9A in the chromatographic membrane carrier 9, the antibody 3241 was used in place of the antibody 3707

### 5. Performance Test

### 5-1. Experiment of Detection of Cultured Measles Viruses

An experiment of detecting measles viruses in a sample was carried out using the test devices 1 and 2 prepared in 4.

In this experiment, two kinds of wild-type measles viruses shown in Table 5 below were cultured in Vero/SLAM cells, and the obtained viruses were diluted with a sodium phosphate buffer containing 1% BSA to give a viral solution. Both the IC-B strain and Edmonston strain are wild-type measles virus strains, and an amino acid sequence derived from the IC-B strain is as shown in SEQ ID NO: 10. An amino acid sequence derived from the Edmonston strain is as shown in SEQ ID NO: 9.

The concentration of the virus in the vial solution was quantified by a plaque method, and its determined concentration is expressed in PFU. The concentration of the virus in each viral solution is as shown in Table 5. PFU (plaque-forming unit) is a unit indicative of the number of viruses calculated from the number of virus-infected cells, which can be confirmed by plaques produced by virus-infected cells prepared by infecting 100% confluent cells with the virus. In Table 5, PFU is expressed in logarithm.

Then, 150 μL of the viral solution containing the virus at the predetermined concentration was added to and mixed with 800 μL of a phosphate buffer, pH 7.3, containing 0.3 w/v % NP-40 (polyoxyethylene (9) octylphenyl ether)), to prepare an antigen sample.

About 200 μL of this antigen sample was dropped onto a glass test tube, and the upstream side (sample receiving member 1) of the test device 1 prepared by the method described above was dipped for 10 minutes in this glass test tube. Thereafter, the coloration with the latex particles in the judgment zone 9A was visually judged. In this judgment, (+) was given where coloration was recognized, and (−) was given where coloration was not recognized. The results are shown in Table 5.

TABLE 5

| | | Test device 1 | Test device 2 | |
|---|---|---|---|---|
| Virus | | MV2-2649 | MV2-2649 | Capturing antibody 1 |
| Name of | | MV2-3707 | MV2-3241 | Capturing antibody 2 |
| virus strain | Log (PFU/mL) | MV3-320 | | Labeled antibody |
| IC-B | 2.60E+04 | + | + | Reactivity |
| | 1.30E+04 | + | + | |
| | 6.40E+03 | + | + | |
| | 3.20E+03 | + | − | |
| | 1.60E+03 | + | − | |
| | 8.00E+02 | − | − | |
| Edmnston | 1.60E+04 | + | + | |
| | 7.90E+03 | + | + | |
| | 4.00E+03 | + | − | |
| | 2.00E+03 | − | − | |

As shown in Table 5, the judgment zone 9A in both the test devices 1 and 2 was recognized to be colored when the viral concentration of the antigen sample was not lower than the predetermined concentration, thus successfully detecting the measles viruses. From the foregoing, it was proven that the test kit for membrane assay in this embodiment can detect measles viruses accurately, and also that the wild-type measles viruses can also be detected.

### 5-2. Experiment of Detecting Measles Viruses in Patient's Analytes

An experiment of detecting measles viruses in analytes collected form patients was conducted using the test devices 1 and 2 prepared in 4.

In 5-1, the test device obtained by applying two kinds of antibody solutions onto different positions in the judgment zone 9A was used, while in this experiment, a test device obtained by applying a mixture of two kinds of antibody solutions onto the judgment zone 9A was used.

From each patient judged to be measles infection-positive through screening by RT-PCR, a throat swab was collected with a cotton-tipped swab. The throat swab was dipped in 75 μL of 1 w/v % SDS solution and thereby eluted into the SDS solution.

The viral concentration of the solution was calculated by comparing Ct value determined by real-time PCR, with CT value of a sample of known concentration quantified by the method described in 5-1.

Then, the above SDS solution was added to and mixed with 800 μL of a phosphate buffer, pH 7.3, containing 0.3 w/v % NP-40 (polyoxyethylene (9) octylphenyl ether)), to prepare an antigen sample.

Using this antigen sample, the coloration with the latex particles in the judgment zone 9A was judged according to the procedure and evaluation criteria described in 5-1. These judgment results are shown in Table 6.

TABLE 6

| | | Test device 1 | Test device 2 | |
|---|---|---|---|---|
| Virus | | MV2-2649 | MV2-2649 | Capturing antibody 1 |
| Name of | | MV2-3707 | MV2-3241 | Capturing antibody 2 |
| virus strain | Log (PFU/mL) | MV3-320 | | Labeled antibody |
| Analyte 1 | 1.31E+00 | + | + | Reactivity |
| Analyte 2 | 3.67E+00 | + | + | |
| Analyte 3 | 1.23E+00 | + | + | |
| Analyte 4 | 2.76E+00 | + | + | |
| Analyte 5 | 4.62E+00 | + | + | |
| Analyte 6 | 4.16E+00 | + | + | |

As shown in Table 6, both the test devices 1 and 2 prepared in this example can be used to detect measles viruses in any of the patients' analytes.

From this result, it has been proven that measles viruses in patients' analyte can be detected accurately with the test devices in this example. It has been also proven from this result that an analyte is collected from a patient to prepare a measurement sample, and this measurement sample and the test device for membrane assay can be used to detect measles viruses by an easy method of judgment with the naked eye.

### 6. Epitope Analysis

That antigen recognition site of the measles virus nuclear protein which was recognized by the anti-measles virus monoclonal antibody in this example was confirmed in the following experiment.

### 6-1. Preparation of Measles Virus Site-Defective Nuclear Proteins

A glutathione S-transferase (GST) fusion protein expression vector pGEX-4T-3 (manufactured by GE Healthcare) was subjected to polymerase chain reaction (PCR) with a primer of SEQ ID NO: 1 containing a recognition sequence with a restriction enzyme NotI (referred to hereinafter as NotI sequence) and a primer of SEQ ID NO: 2 containing a recognition sequence with a restriction enzyme BamHI (referred to hereinafter as BamHI sequence), whereby a cDNA having the NotI sequence and the BamHI sequence integrated upstream and downstream of the GST sequence respectively was synthesized.

The synthesized cDNA was treated with restriction enzymes NotI and BamHI. Similarly, a vector attached to a cell-free wheat germ expression reagent kit ENDEXT (registered trade mark) Wheat Germ Expression TRI-GG Kit (Cell Free Science) was treated with restriction enzymes NotI and BamHI.

The resulting restriction enzyme-treated products were ligated to each other to construct a cell-free wheat germ expression vector containing the GST sequence.

From RNA extracted from the measles virus Edomonston strain, cDNA was synthesized by reverse transcription reaction. The synthesized measles virus cDNA was subjected to PCR with a primer of SEQ ID NO: 3 containing a recognition sequence with a restriction enzyme XhoI (referred to hereinafter as XhoI sequence) and a primer of SEQ ID NO: 4. The resulting PCR product was then subjected as a template to PCR with the primer of SEQ ID NO: 3 and a primer of SEQ ID NO: 5 containing a NotI sequence. A DNA product containing the XhoI sequence and the NotI sequence upstream and downstream of the measles virus nuclear protein respectively was synthesized.

The synthesized DNA product was treated with restriction enzymes XhoI and NotI. Similarly, the cell-free wheat germ expression vector containing the GST sequence was treated with restriction enzymes XhoI and NotI. The resulting restriction enzyme products were ligated to each other to construct a cell-free wheat germ expression vector containing the measles virus nuclear protein sequence and the GST sequence (referred to hereinafter as “full-length nuclear protein expression vector”). From the full-length nuclear protein expression vector thus constructed, a full-length nuclear protein was expressed with the cell-free wheat germ expression reagent kit ENDEXT (registered trade mark) Wheat Germ Expression TRI-GG Kit (Cell Free Science).

For analyzing those antigen recognition sites of the measles virus nuclear protein that were recognized by the antibody 2649, antibody 3707, antibody 3241 and antibody 320, site-defective nuclear protein truncate-1 to truncate-4 derived from the full-length measles virus nuclear protein by removing its C terminal were prepared by the following method. The prepared site-defective nuclear proteins are schematically shown in FIG. 9.

On the basis of the full-length nuclear protein expression vector, the whole circumference of the plasmid excluding amino acids between the position 399 and the C terminal (position 525) of the measles virus nuclear protein was amplified with a primer of SEQ ID NO: 6, a primer of SEQ ID NO: 7, and KOD plus Mutagenesis Kit (manufactured by Toyobo), whereby a truncate-1 expression vector was constructed. From the constructed truncate-1 expression vector, truncate-1 was expressed with the cell-free wheat germ expression reagent kit ENDEXT (registered trade mark) Wheat Germ Expression TRI-GG Kit (Cell Free Science). The nuclear protein from which the amino acids between the position 399 and the C terminal had been deleted was thereby obtained.

Truncate-2 was obtained by the same method as for truncate-1 except that a primer of SEQ ID NO: 8 was used in place of the primer of SEQ ID NO: 7. The nuclear protein from which the amino acids between the position 321 and the C terminal of the measles virus nuclear protein had been deleted was thereby obtained.

Truncate-3 was obtained by the same method as for truncate-1 except that a primer of SEQ ID NO: 9 was used in place of the primer of SEQ ID NO: 7. The nuclear protein from which the amino acids between the position 242 and the C terminal of the measles virus nuclear protein had been deleted was thereby obtained.

Truncate-3 was obtained by the same method as for truncate-1 except that a primer of SEQ ID NO: 10 was used in place of the primer of SEQ ID NO: 7. The nuclear protein from which the amino acids between the position 135 and the C terminal of the measles virus nuclear protein had been deleted was thereby obtained.

Then, truncate-5 from which the amino acids between the N terminal of the nuclear protein and the position 398 was prepared.

Truncate-5 was obtained by the same method as in preparation of the full-length nuclear expression vector and in expression of the vector except that a primer of SEQ ID NO: 11 was used in place of the primer of SEQ ID NO: 3. The primer of SEQ ID NO: 11 is an XhoI sequence-containing primer corresponding to a nucleotide sequence consisting of nucleotide 1195 and its downstream nucleotides in the cDNA of the measles virus nuclear protein.

For analyzing those antigen recognition sites of the measles virus nuclear protein that were recognized by the antibody 2649, antibody 3707, antibody 3241 and antibody 320 in more detail, site-defective nuclear protein truncate-6 to truncate-14 were prepared. The prepared site-defective nuclear proteins are schematically shown in FIG. 9.

On the basis of the full-length nuclear protein expression vector, the whole circumference of the plasmid excluding amino acids between the N terminal and the position 44 of the measles virus nuclear protein was amplified with a primer of SEQ ID NO: 12, a primer of SEQ ID NO: 13, and KOD plus Mutagenesis Kit (manufactured by Toyobo), whereby a truncate-6 expression vector was constructed. From the constructed truncate-6 expression vector, truncate-6 was expressed with the cell-free wheat germ expression reagent kit ENDEXT (registered trade mark) Wheat Germ Expression TRI-GG Kit (Cell Free Science) The nuclear protein from which the amino acids between the N terminal and the position 44 had been deleted was thereby obtained.

Truncate-7 was obtained by the same method as for truncate-6 except that a primer of SEQ ID NO: 14 was used in place of the primer of SEQ ID NO: 12 and a primer of SEQ ID NO: 15 was used in place of the primer of SEQ ID NO: 13. The nuclear protein from which the amino acids from the position 45 to position 90 of the measles virus nuclear protein had been deleted was thereby obtained.

Truncate-8 was obtained by the same method as for truncate-6 except that a primer of SEQ ID NO: 16 was used in place of the primer of SEQ ID NO: 12 and a primer of SEQ ID NO: 17 was used in place of the primer of SEQ ID NO: 13. The nuclear protein from which the amino acids from the position 91 to position 134 of the measles virus nuclear protein had been deleted was thereby obtained.

Truncate-9 was obtained by the same method as for truncate-6 except that a primer of SEQ ID NO: 18 was used in place of the primer of SEQ ID NO: 12 and a primer of SEQ ID NO: 10 was used in place of the primer of SEQ ID NO: 13. The nuclear protein from which the amino acids from the position 135 to position 170 of the measles virus nuclear protein had been deleted was thereby obtained.

Truncate-10 was obtained by the same method as for truncate-6 except that a primer of SEQ ID NO: 19 was used in place of the primer of SEQ ID NO: 12 and a primer of SEQ ID NO: 20 was used in place of the primer of SEQ ID NO: 13. The nuclear protein from which the amino acids from the position 171 to position 206 of the measles virus nuclear protein had been deleted was thereby obtained.

Truncate-11 was obtained by the same method as for truncate-6 except that a primer of SEQ ID NO: 21 was used in place of the primer of SEQ ID NO: 12 and a primer of SEQ ID NO: 22 was used in place of the primer of SEQ ID NO: 13. The nuclear protein from which the amino acids from the position 207 to position 241 of the measles virus nuclear protein had been deleted was thereby obtained.

Truncate-12 was obtained by the same method as for truncate-6 except that a primer of SEQ ID NO: 23 was used in place of the primer of SEQ ID NO: 12 and a primer of SEQ ID NO: 7 was used in place of the primer of SEQ ID NO: 13. The nuclear protein from which the amino acids from the position 399 to position 441 of the measles virus nuclear protein had been deleted was thereby obtained.

Truncate-13 was obtained by the same method as for truncate-6 except that a primer of SEQ ID NO: 24 was used in place of the primer of SEQ ID NO: 12 and a primer of SEQ ID NO: 25 was used in place of the primer of SEQ ID NO: 13. The nuclear protein from which the amino acids from the position 442 to position 484 of the measles virus nuclear protein had been deleted was thereby obtained.

Truncate-14 was obtained by the same method as for truncate-6 except that a primer of SEQ ID NO: 6 was used in place of the primer of SEQ ID NO: 12 and a primer of SEQ ID NO: 26 was used in place of the primer of SEQ ID NO: 13. The nuclear protein from which the amino acids from the position 485 to position 525 of the measles virus nuclear protein had been deleted was thereby obtained.

The resulting full-length nuclear protein and the truncates 1 to 14 were purified as GST-bound proteins by means of glutathione Sepharose 4B 50% (manufactured by GE Healthcare).

For a negative control, a GST sequence-added plasmid was integrated in a vector attached to the cell-free wheat germ expression kit, then transcribed and translated, and the resulting protein was purified. The resulting protein was used as a negative control for the antigen recognition site.

### 6-2. Analysis of the Antigen Recognition Sites

The antigen solutions of the full-length nuclear protein and the truncates 1 to 14 prepared in 6-1 above were diluted respectively with the second buffer solution to prepare antigen samples.

A microtiter plate to which an anti-GST antibody (manufactured by GE Healthcare) had been conjugated was prepared in the following manner.

The anti-GST antibody solution was diluted to a concentration of 5 μg/mL with 0.1 M phosphate buffer solution (PBS, pH 7.5) containing 0.1 w/v % NaN₃. The resulting anti-GST antibody dilution was pipetted in a volume of 50 μL to each well of a microtiter plate (manufactured by NUNC) and left overnight at 4° C. Each well was washed 3 times with the first buffer solution, and then 300 μL of the second buffer solution was pipetted into each well, thereby blocking the antibody (hereinafter, this microtiter plate will be referred to as anti-GST antibody-conjugated plate). After blocking, the plate was left at 2 to 8° C. for 4 hours or more in a stationary state. The anti-GST antibody-conjugated plate was stored at 2 to 8° C. until use.

The second buffer solution in the anti-GST antibody-conjugated plate was removed. After removal, 50 μL antigen solution (the full-length nuclear protein or each of truncates 1 to 14) was added to each well of the anti-GST antibody-conjugated plate and then stirred for 30 minutes. Thereafter, each well of the anti-GST antibody-conjugated plate was washed 3 times with the second buffer solution.

Then, the antibody 2649, antibody 3707, antibody 3241 and antibody 320 were diluted to 10 μg/mL with the second buffer solution to prepare the respective antibody reaction solutions. 50 μL of each antibody reaction solution was added to each well of the anti-GST antibody-conjugated plate and stirred for 30 minutes. Thereafter, each well of the anti-GST antibody-conjugated plate was washed 3 times with the second buffer solution.

A peroxidase (POD)-bound anti-mouse IgG antibody (manufactured by DAKO Cytomation) was diluted to 20 mU/mL with the second buffer solution to prepare a POD-bound anti-mouse IgG antibody solution. 50 μL of the POD-bound anti-mouse IgG antibody solution was added to each well of the anti-GST antibody-conjugated plate and stirred for 30 minutes. Thereafter, each well of the anti-GST antibody-conjugated plate was washed 3 times with the second buffer solution.

Then, 100 μL of a substrate solution containing OPD was added to each well of the anti-GST antibody-conjugated plate and then left at room temperature for 10 minutes. Then, 100 μL of a reaction termination solution (containing 2 N H₂SO₄) was added to each well of the anti-GST antibody-conjugated plate, and the reaction solution in each well was measured for its absorbance at 492 nm with a microplate reader.

The obtained absorbances are shown in Table 7, and the evaluation results, based on absorbance, of each antibody for each antigen (the full-length nuclear protein or each of truncates 1 to 14) are shown in Table 8. In Table 8, “+++” indicates that the absorbance is 1.5 or more; “++”, 0.5 and or more and less than 1.5; “+”, the 3SD value of the negative control or more and less than 0.5; and “−”, the 3SD of the negative control or less.

TABLE 7

| | | Antigen | | | | | | |
|---|---|---|---|---|---|---|---|---|
| | | Full | | | | | | |
| Antibody | | length | | | | | | Antigen |
| Clone | | No | Truncate-1 | Truncate-2 | Truncate-3 | Truncate-4 | Truncate-5 | name |
| name | Isotype | defect | 399-525 | 321-525 | 242-525 | 135-525 | 1-398 | Defect site |
| MV2-2649 | IgG1/κ | 3.476 | 3.215 | 1.636 | 3.454 | 0.038 | 0.203 | Absorbance |
| MV2-3707 | IgG1/κ | 3.678 | 0.014 | 0.012 | 0.015 | 0.017 | 3.580 | |
| MV2-3241 | IgG2a/κ | 3.882 | 0.165 | 0.127 | 0.121 | 0.021 | 3.749 | |
| MV3-320 | IgG2b/κ | 3.412 | 3.405 | 3.643 | 3.533 | 2.543 | 0.009 | |
| | | Antigen | | | | | | |
| Antibody | | | | | | Truncate- | Truncate- | Antigen |
| Clone | | Truncate-6 | Truncate-7 | Truncate-8 | Truncate-9 | 10 | 11 | name |
| name | Isotype | 1-44 | 45-90 | 91-134 | 135-170 | 171-206 | 207-241 | Defect site |
| MV2-2649 | IgG1/κ | 0.245 | 0.249 | 0.210 | 0.201 | 0.255 | 0.236 | Absorbance |
| MV2-3707 | IgG1/κ | 3.580 | 3.670 | 3.537 | 3.423 | 3.555 | 3.502 | |
| MV2-3241 | IgG2a/κ | 3.787 | 3.933 | 3.735 | 3.583 | 3.731 | 3.655 | |
| MV3-320 | IgG2b/κ | 0.724 | 0.892 | 0.016 | 0.475 | 3.307 | 3.237 | |

| | | Antigen | | | |
|---|---|---|---|---|---|
| | | | | | Antigen |
| Antibody | | | | | name |
| Clone | | Truncate-12 | Truncate-13 | Truncate-14 | Defect |
| name | Isotype | 399-441 | 442-484 | 485-525 | site |
| MV2-2649 | IgG1/κ | 3.399 | 2.626 | 2.235 | Absorbance |
| MV2-3707 | IgG1/κ | 3.444 | 2.596 | 0.012 | |
| MV2-3241 | IgG2a/κ | 3.555 | 2.689 | 0.130 | |
| MV3-320 | IgG2b/κ | 3.264 | 2.195 | 2.252 | |

| | |
|---|---|
| | No antigen |
| Antibody | Cell-free wheat germ |

| | |
|---|---|
| Clone | expression vector only |

| name | Isotype | Absorbance | 3SD |
|---|---|---|---|
| MV2-2649 | IgG1/κ | 0.031 | 0.050 |
| MV2-3707 | IgG1/κ | 0.012 | 0.016 |
| MV2-3241 | IgG2a/κ | 0.012 | 0.015 |
| MV3-320 | IgG2b/κ | 0.013 | 0.019 |

TABLE 8

| | | Antigen | | | | | | |
|---|---|---|---|---|---|---|---|---|
| | | Full | | | | | | |
| Antibody | | length | | | | | | Antigen |
| Clone | | No | Truncate-1 | Truncate-2 | Truncate-3 | Truncate-4 | Truncate-5 | name |
| name | Isotype | defect | 399-525 | 321-525 | 242-525 | 135-525 | 1-398 | Defect site |
| MV2-2649 | IgG1/κ | +++ | +++ | +++ | +++ | − | + | Reactivity |
| MV2-3707 | IgG1/κ | +++ | − | − | − | − | +++ | |
| MV2-3241 | IgG2a/κ | +++ | + | + | + | − | +++ | |
| MV3-320 | IgG2b/κ | +++ | +++ | +++ | +++ | +++ | − | |
| | | Antigen | | | | | | |
| Antibody | | | | | | Truncate- | Truncate- | Antigen |
| Clone | | Truncate-6 | Truncate-7 | Truncate-8 | Truncate-9 | 10 | 11 | name |
| name | Isotype | 1-44 | 45-90 | 91-134 | 135-170 | 171-206 | 207-241 | Defect site |
| MV2-2649 | IgG1/κ | + | + | + | + | + | + | Reactivity |
| MV2-3707 | IgG1/κ | +++ | +++ | +++ | +++ | +++ | +++ | |
| MV2-3241 | IgG2a/κ | +++ | +++ | +++ | +++ | +++ | +++ | |
| MV3-320 | IgG2b/κ | ++ | ++ | − | + | +++ | +++ | |

| | | Antigen | | | |
|---|---|---|---|---|---|
| | | | | | Antigen |
| Antibody | | | | | name |
| Clone | | Truncate-12 | Truncate-13 | Truncate-14 | Defect |
| name | Isotype | 399-441 | 442-484 | 485-525 | site |
| MV2-2649 | IgG1/κ | +++ | +++ | +++ | Reactivity |
| MV2-3707 | IgG1/κ | +++ | +++ | − | |
| MV2-3241 | IgG2a/κ | +++ | +++ | + | |
| MV3-320 | IgG2b/κ | +++ | +++ | +++ | |

From the results in Tables 7 and 8, the 2649 antibody was suggested to recognize an amino acid sequence of the residue 135 to the residue 241. The 3707 and 3241 antibodies were suggested to recognize an amino acid sequence of the residue 485 to the residue 525. Further, the 320 antibody was suggested to recognize an amino acid sequence of the residue 91 to the residue 134.

What is claimed is:

1. Method for detecting a measles virus in an analyte, comprising:
forming a complex of a first monoclonal antibody being capable of binding to a first epitope of a measles virus nuclear protein and being immobilized on a solid phase, a second monoclonal antibody being capable of binding to a second epitope of a measles virus nuclear protein different from the first epitope and being labeled, and a measles virus nuclear protein contained in the analyte, on the solid phase; and
detecting the measles virus based on the amount of the label of the complex formed on the solid phase.

2. The method of claim 1, further comprises a step of preparing a measurement sample by mixing an analyte with an analyte treatment liquid containing a nonionic surfactant,
wherein the forming step comprises a step of forming the complex by using the prepared measurement sample.

3. The method of claim 1, wherein the analyte is selected from the group consisting of sputum, a nasal discharge, a nasal cavity swab, a nasal cavity aspirate and a throat swab.

4. The method of claim 1, wherein the first monoclonal antibody and the second monoclonal antibody are selected from the group consisting of:
a monoclonal antibody capable of binding to amino acid sequences of the residue 135 to the residue 241 of a measles virus nuclear protein and binding to the amino;
a monoclonal antibody capable of binding to amino acid sequences of the residue 485 to the residue 525 of a measles virus nuclear protein; and
a monoclonal antibody capable of recognizing amino acid sequences of the residue 91 to the residue 134 of a measles virus nuclear protein.

5. The method of claim 4, wherein the second monoclonal antibody is a monoclonal antibody capable of recognizing amino acid sequences of the residue 91 to the residue 134 of a measles virus nuclear protein.

6. A test device for a membrane assay comprising:
a membrane carrier containing a judgment zone that holds a first monoclonal antibody capable of binding to first epitope of a measles virus nuclear protein; and
a label holding member that holds a second monoclonal antibody being capable of binding to second epitope of a measles virus nuclear protein different from the first epitope and being labeled.

7. The test device of claim 6, wherein the judgment zone further holds a third monoclonal antibody being capable of binding to third epitope of a measles virus nuclear protein different from the first and second epitope.

8. A test kit for a membrane assay comprising:
the test device for a membrane assay of claim 6; and
an analyte treatment liquid containing a nonionic surfactant and being mixed with an analyte to prepare a measurement sample.

9. The test kit of claim 8, further comprises a test container being capable of accommodating the measurement sample and being constituted such that the test device can be inserted into it.

10. A test kit for a membrane assay comprising:
a test device provided with a membrane carrier containing a judgment zone holding a first monoclonal antibody capable of binding to first epitope of a measles virus nuclear protein; and
a label solution containing a second monoclonal antibody being capable of binding to second epitope of a measles virus nuclear protein different from the first epitope and being labeled.

11. The test kit of claim 10, wherein the judgment zone further holds a third monoclonal antibody being capable of binding to third epitope of a measles virus nuclear protein different from the first and second epitope.

12. The test kit of claim 10, wherein the label solution further comprises a nonionic surfactant.

13. A hybridoma selected from the group consisting of hybridomas deposited under Accession Nos. NITE BP-563, NITE BP-564, NITE BP-565 and NITE BP-566 with NITE Patent Microorganisms Depositary, National Institute of Technology and Evaluation (NITE), National Institute of Advanced Industrial Science and Technology, Japan.

14. A monoclonal antibody produced by the hybridoma of claim 13.


SEQUENCE LISTING (36 sequences)

SEQ ID NO: 1 - protein, 542 aa, Measles virus
MATLLRSLALFKRNKDKPPITSGSGGAIRGIKHIIIVPIPGDSSITTRSRLLDRLVRLIGNPDVSGPKLTGALIGILSLFVESPGQLIQRITDDPDVSIRLLEVVQSDQSQSGLTFASRGTNMEDEADQYFSHDDPSSGDQSRFGWFENKEISDIEVQDPEGFNMILGTILAQIWVLLAKAVTAPDTAADSELRRWIKYTQQRRVVGEFRLERKWLDVVRNRIAEDLSLRRFMVALILDIKRTPGNKPRIAEMICDIDTYIVEAGLASFILTIKFGIETMYPALGLHEFAGELSTLESLMNLYQQMGETAPYMVILENSIQNKFSAGSYPLLWSYAMGVGVELENSMGGLNFGRSYFDPAYFRLGQEMVRRSAGKVSSTLASELGITAEDARLVSEIAMHTTEDRTSRAVGPRQAQVSFLHGDQSENELPGLGGKEDRRVKQSRGETRENSRETGPRRASDARAAHLPTSTPPDIDTASEYSQEPQDSRRSADALLRLQAMAGILEEQGSDTDTPRVYNDRDLLDAVLEENLYFQGSFSGRM

SEQ ID NO: 2 - protein, 542 aa, Measles virus
MATLLRSLALFKRNKDKPPITSGSGGAIRGIKHIIIVPIPGDSSITTRSRLLDRLVRLIGNPDVSGPKLTGALIGILSLFVESPGQLIQRITDDPDVSIRLLEVVQSDQSQSGLTFASRGTNMEDEADQYFSHDDPSSSDQSRSGWFENKEISDIEVQDPEGFNMILGTILAQIWVSLAKAVTAPDTAADSELRRWIKYTQQRRVVGEFRLERKWLDVVRNRIAEDLSLRRFMVALILDIKRTPGNKPRIAEMICDIDTYIVEAGLASFILTIKFGIETMYPALGLHEFAGELSTLESLMNLYQQMGETAPYMVILENSIQNKFSAGSYPLLWSYAMGVGVELENSMGGLNFGRSYFDPAYFRLGQEMVRRSAGKVSSTLASELGITAEDARLVSEIAMHTTEDRISRAVGPRQAQVSFLHGDQSENELPGLGGKEDRRVKQGRGEARESYRETGSSRASDARAAHPPTSMPLDIDTASESGQDPQDSRRSADALLRLQAMAGILEEQGSDTDTPRVYNDRDLLDAVLEENLYFQGSFSGRM

SEQ ID NO: 3 - protein, 542 aa, Measles virus
MATLLRSLALFKRNKDKPPITSGSGGAIRGIKHIIIVPIPGDSSITTRSRLLDRLVRLIGNPDVSGPKLTGALIGILSLFVESPGQLIQRITDDPDVSIRLLEVVQSDQSQSGLTFASRGTNMEDEADQYFSHDDPSSGDQSRFGWFENKEISDIEVQDPEGFNMILGTILAQIWVLLAKAVTAPDTAADSELRRWIKYTQQRRVVGEFRLERKWLDVVRNRIAEDLSLRRFMVALILDIKRTPGNKPRIAEMICDIDTYIVEAGLASFILTIKFGIETMYPALGLHEFAGELSTLESLMNLYQQMGETAPYMVILENSIQNKFSAGSYPLLWSYAMGVGVELENSMGGLNFGRSYFDPAYFRLGQEMVRRSAGKVSSTLASELGITAEDARLVSEIAMHTTEDRTSRAVGPRQAQVSFLHGDQSENELPGLGGREDRRVKQSRGETRENSRETGPRRASDARAAHLPTSTPPDIDTASEYSQEPQDSRRSADALLRLQAMAGILEEQGSDTDTPRVYNDRDLLDAVLEENLYFQGSFSGRM

SEQ ID NO: 4 - protein, 542 aa, Measles virus
MATLLRSLALFKRNKDKPPITSGSGGAIRGIKHIIIVPIPGDSSITTRSRLLDRLVRLIGNPDVSGPKLTGALIGILSLFVESPGQLIQRITDDPDVSIRLLEVVQSDQSQSGLTFASRGTNMEDEADQYFSHDDPSSGDQSRFGWFENKEISDIEVQDPEGFNMILGTILAQIWVLLAKAVTAPDTAADSELRRWIKYTQQRRVVGEFRLERKWLDVVRNRIAEDLSLRRFMVALILDIKRTPGNKPRIAEMICDIDTYIVEAGLASFILTIKFGIETMYPALGLHEFAGELSTLESLMNLYQQMGETAPYMVILENSIQNKFSAGSYPLLWSYAMGVGVELENSMGGLNFGRSYFDPAYFRLGQEMVRRSAGKVSSTLASELGITAEDARLVSEIAMHTTEDRTSRAVGPRQAQVSFLHGDQSENELPGLGGKEDRRVKQSRGETRENSRETGPRRASDARAAHLPTSTPLDIDTASEYSQEPQDSRRSADALLRLQAMAGILEEQGSDTDTPRVYNDRDLLDAVLEENLYFQGSFSGRM

SEQ ID NO: 5 - protein, 542 aa, Measles virus; identical to SEQ ID NO: 1

SEQ ID NO: 6 - protein, 542 aa, Measles virus
MATLLRSLALFKRNKDKPPITSGSGGAIRGIKHIIIVPIPGDSSITTRSRLLDRLVRLIGNPDVSGPKLTGALIGILSLFVESPGQLIQRITDDPDVSIRLLEVVQSDQSQSGLTFASRGTNMEDEADQYFSHDDPSSGDQSRFGWFENKEISDIEVQDPEGFNMILGTILAQIWVLLAKAVTAPDTAADSELRRWIKYTQQRRVVGEFRLERKWLDVVRNRIAEDLSLRRFMVALILDIKRTPGNKPRIAEMICDIDTYIVEAGLASFILTIKFGIETMYPALGLHEFAGELSTLESLMNLYQQMGETAPYMVILENSIQNKFSAGSYPLLWSYAMGVGVELENSMGGLNFGRSYFDPAYFRLGQEMVRRSAGKVSSTLASELGITAEDARLVSEIAMHTTEDRTSRAVGPRQAQVSFLHGDQSENELPGLGGKEDGRVKQSRGETRENSRETGPRRASDARAAHLPTSTPPDIDTASEYSQEPQDSRRSADALLRLQAMAGILEEQGSDTDTPRVYNDRDLLDAVLEENLYFQGSFSGRM

SEQ ID NO: 7 - protein, 542 aa, Measles virus
MATLLRSLALFKRNKDKPPITSGSGGAIRGIKHIIIVPIPGDSSITTRSRLLDRLVRLIGNPDVSGPKLTGALIGILSLFVESPGQLIQRITDDPDVSIRLLEVVQSDQSQSGLTFASRGTNMEDEADQYFSHDDPSSSDQSGSGWFENKEISDIEVQDPEGFNMILGTILAQIWVLLAKAVTAPDTAADSELRRWIKYTQQRRVVGEFRLERKWLDVVRNRIAEDLSLRRFMVALILDIKRTPGNKPRIAEMICDIDTYIVEAGLASFILTIKFGIETMYPALGLHEFAGELSTLESLMNLYQQMGETAPYMVILENSLQNKFSAGSYPLLWSYAMGVGVELENSMGGLNFGRSYFDPAYFRLGQEMVRRSAGKVSSTLASELGITAEDARLVSEIAMHTTEDRISRAVGPRQAQVSFLHGDQSENELPGLGGKEDRRVKQSRGEARESYRETGSSRASDARAAHLPTSTPLDIDTASESGQDPQDSRRSADALLRLQAMAGILEEQGSDTDTPKVYNDRDLLDAVLEENLYFQGSFSGRM

SEQ ID NO: 8 - protein, 542 aa, Measles virus
MATLLRSLALFKRNKDKPPITSGSGGAIRGIKHIIIVPIPGDSSITTRSRLLDRLVRLIGNPDVSGPKLTGALIGILSLFVESPGQLIQRITDDPDVSIRLLEVVQSDQSQSGLTFASRGTNMEDEADQYFSHDDPSGSDQSRSGWFENKEISDIEVQDPEGFNMILGTILAQIWVLLAKAVTAPDTAADSELRRWIKYTQQRRVVGEFRLERKWLDVVRNRIAEDLSLRRFMVALILDIKRTPGNKPRIAEMICDIDTYIVEAGLASFILTIKFGIETMYPALGLHEFAGELSTLESLMNLYQQMGETAPYMVILENSIQNKFSAGSYPLLWSYAMGVGVELENSMGGLNFGRSYFDPAYFRLGQEMVRRSAGKVSSTLASELGITAEDARLVSEIAMHTTEDRISRAVGPRQAQVSFLHGDQSENELPGLGGKEDRRVKQSRGEARESYRETGSSRASDERAAHLPTSTPLDIDTASESGQDPQDSRRSADALLRLQAMAGILEEQGSDTDTPRVYNDRDLLDAVLEENLYFQGSFSGRM

SEQ ID NO: 9 - protein, 525 aa, Measles virus
MATLLRSLALFKRNKDKPPITSGSGGAIRGIKHIIIVPIPGDSSITTRSRLLDRLVRLIGNPDVSGPKLTGALIGILSLFVESPGQLIQRITDDPDVSIRLLEVVQSDQSQSGLTFASRGTNMEDEADKYFSHDDPISSDQSRFGWFENKEISDIEVQDPEGFNMILGTILAQIWVLLAKAVTAPDTAADSELRRWIKYTQQRRVVGEFRLERKWLDVVRNRIAEDLSLRRFMVALILDIKRTPGNKPRIAEMICDIDTYIVEAGLASFILTIKFGIETMYPALGLHEFAGELSTLESLMNLYQQMGETAPYMVILENSIQNKFSAGSYPLLWSYAMGVGVELENSMGGLNFGRSYFDPAYFRLGQEMVRRSAGKVSSTLASELGITAEDARLVSEIAMHTTEDKISRAVGPRQAQVSFLHGDQSENELPRLGGKEDRRVKQSRGEARESYRETGPSRASDARAAHLPTGTPLDIDTASESSQDPQDSRRSADALLRLQAMAGISEEQGSDTDTPIVYNDRNLLD

SEQ ID NO: 10 - protein, 525 aa, Measles virus; identical to SEQ ID NO: 9

SEQ ID NO: 11 - DNA, 35 bp, Artificial Sequence
  Primer
tttgcggccgcatgtcccctatactaggttattgg

SEQ ID NO: 12 - DNA, 34 bp, Artificial Sequence
  Primer
tttggatcctcaatccgattttggaggatggtcg

SEQ ID NO: 13 - DNA, 31 bp, Artificial Sequence
  Primer
gccgctcgagatggccacacttttaaggagc

SEQ ID NO: 14 - DNA, 56 bp, Artificial Sequence
  Primer
cctgaaaatacaggttttcttcaagtacggcgtctagaagatttctgtcattgtac

SEQ ID NO: 15 - DNA, 44 bp, Artificial Sequence
  Primer
tttgcggccgctgaaggagccctgaaaatacaggttttcttcaa

SEQ ID NO: 16 - DNA, 21 bp, Artificial Sequence
  Primer
gccgtacttgaagaaaacctg

SEQ ID NO: 17 - DNA, 22 bp, Artificial Sequence
  Primer
tgcaatctctgaaacaagcctt

SEQ ID NO: 18 - DNA, 23 bp, Artificial Sequence
  Primer
aattgagttctccaggattacca

SEQ ID NO: 19 - DNA, 27 bp, Artificial Sequence
  Primer
gaattcgatatccaggattagagcgac

SEQ ID NO: 20 - DNA, 23 bp, Artificial Sequence
  Primer
atcatgtgaaaagtatttgtccg

SEQ ID NO: 21 - DNA, 34 bp, Artificial Sequence
  Primer
gggggctcgagatgcatactactgaggacaagat

SEQ ID NO: 22 - DNA, 22 bp, Artificial Sequence
  Primer
attaccactcgatccagacttc

SEQ ID NO: 23 - DNA, 22 bp, Artificial Sequence
  Primer
catctcgagaactagtgatatc

SEQ ID NO: 24 - DNA, 20 bp, Artificial Sequence
  Primer
atcaccgatgaccctgacgt

SEQ ID NO: 25 - DNA, 21 bp, Artificial Sequence
  Primer
tgaggaatctccagggattgg

SEQ ID NO: 26 - DNA, 23 bp, Artificial Sequence
  Primer
gatccaattagtagtgatcaatc

SEQ ID NO: 27 - DNA, 21 bp, Artificial Sequence
  Primer
cctctgaatcaattgacctgg

SEQ ID NO: 28 - DNA, 21 bp, Artificial Sequence
  Primer
ctagcccaaatttgggtcttg

SEQ ID NO: 29 - DNA, 23 bp, Artificial Sequence
  Primer
ggtgaatttagattggagagaaa

SEQ ID NO: 30 - DNA, 22 bp, Artificial Sequence
  Primer
gatggtacccagaatcatgttg

SEQ ID NO: 31 - DNA, 21 bp, Artificial Sequence
  Primer
agaacacccggaaacaaaccc

SEQ ID NO: 32 - DNA, 22 bp, Artificial Sequence
  Primer
aactacccttctttgttgggtg

SEQ ID NO: 33 - DNA, 20 bp, Artificial Sequence
  Primer
cagagtcgaggagaagccag

SEQ ID NO: 34 - DNA, 19 bp, Artificial Sequence
  Primer
ccgcaggacagtcgaaggt

SEQ ID NO: 35 - DNA, 22 bp, Artificial Sequence
  Primer
tttgaccctcctatcttccttg

SEQ ID NO: 36 - DNA, 20 bp, Artificial Sequence
  Primer
atcttggctggactccgatg